(19) United States
(12) Patent Application Publication
(10) Pub. No.: US 2004/0235812 A1
(43) Pub. Date: Nov. 25, 2004

(54) Pharmaceutical composition comprisng an aromatase inhibitor and an estrogen suitable for hormone replacement therapy for a male

(76) Inventor: Robert F Caspers, Toronto (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/476,836

(22) PCT Filed: Apr. 3, 2003
(86) PCT No.: PCT/CA03/00492

Related U.S. Application Data
(60) Provisional application No. 60/369,640, filed on Apr. 3, 2002.

(51) Int. Cl. A61K 31/56

(52) U.S. Cl. 514/182

(57) ABSTRACT

A pharmaceutical preparation for males for increasing physiologic endogenous testosterone comprising at least one aromatase inhbitor and an estrogen and methods of use thereof for increasing male libido are provided.

### FIELD OF THE INVENTION

This invention relates to hormone replacement therapy for a male and more particularly to a pharmaceutical preparation comprising at least one aromatase inhibitor combined with a physiologic replacement dose of estrogen suitable for physiologic hormone replacement therapy in men.

### BACKGROUND OF THE INVENTION

In contrast to a rapid decline of estradiol during menopause in women, the process of reproductive aging in the male is gradual and delayed and subject to wide individual variations. Impairment of spermatogenesis is observed as a continuous process occurring over decades. However, spermatogenesis may be retained well into senescence and only about 50% of men in their eighties show complete loss of fertility, as described in Schill W B. Asian J Androl 2001; 3:1-7. Of greater importance for individual health is altered sex hormone concentrations in aging men that results from both a gradual reduction of, and functional disturbances in, Leydig cells. Furthermore, there may be an impaired feedback mechanism at the level of the pituitary-gonadal axis, with disappearance of the early morning testosterone rise, and increased LH (luteinizing hormone) and FSH (follicle stimulating hormone) levels. Lower total testosterone concentrations in men over 60 years of age are accompanied by clinical signs of reduced virility, such as decreased muscle mass and strength as well as reduced sexual hair growth and libido. Andropause is a term of convenience, which has been used to describe this complex of symptoms in aging men who have low testosterone levels, as described in Bain J., Can Fam Physician 2001; 47: 91-7. An age-related decline in androgen secretion and plasma testosterone levels therefore suggests the use of androgen supplementation. The major androgen target organs of interest with regard to beneficial effects of male HRT (hormone replacement therapy) include bone, muscle, adipose tissue, the cardiovascular system and the central nervous system (libido and aspects of mood), as described in Tenover J L., Int J Androl 1999; 22:300-6. However, there is a lack of long-term risk-benefit studies. In addition, at present, androgen replacement is difficult to control and requires injections of testosterone at regular intervals. Testosterone injection can result in supraphysiologic peaks and fluctuations of serum testosterone, leading to potential side effects involving numerous organ systems. In addition, the injections are usually uncomfortable and require a visit to a clinic for administration. Alternative replacement therapy includes oral and transdermal testosterone, both of which may be associated with adverse effects such as liver damage with oral methyltestosterone and skin irritation with transdermal testosterone. These adverse effects have been shown in studies described in Wu F C. Et al., Fertil Steril 1996; 65:626-36, and Slayden S M., Semin Reprod Endocrinol 1998; 16:145-52. Therefore, a patient-controlled method to consistently increase androgen levels without side effects would be a major advantage.

It is now appreciated that libido in males is dependent on adequate levels of androgen as well as adequate brain concentrations of estrogen, as described in Zumpe D. et al., Physiol Behav 1994; 56:665-9. The need for estrogen to maintain libido has been deduced from studies involving administration of aromatase inhibitors in primates and other species, and from clinical cases of aromatase deficiency, described in Balthazart J. et al., Behav Neurosci 1997; 11 1:381-97, and Carani C. et al., Clin Endocrinol (Oxf) 1999; 51:517-24. In primates treated with an aromatase inhibitor to suppress brain estrogen, libido was markedly reduced despite an increase in testosterone levels. Addition of estrogen did not restore libido in this animal model and the investigators concluded that the estrogen replacement did not reach the brain in adequate concentrations, described in Zumpe D et al. In a clinical case report of a male with decreased libido secondary to congenital deficiency of aromatase and undetectable estrogen levels but normal androgen concentrations, libido was restored by low doses of estrogen replacement, described in Carani C. et al. This study, therefore, suggests that estrogen does enter the brain in humans and can favorably affect libido.

### SUMMARY OF THE INVENTION

The present invention provides a pharmaceutical composition for hormone replacement therapy in a male, the composition comprising an aromatase inhibitor and an estrogen.

The present invention provides a pharmaceutical preparation for administration to a male in need of hormone replacement therapy, comprising a plurality of doses for administration, each dose comprising at least one aromatase inhibitor and an estrogen.

The present invention further provides a package containing a pharmaceutical preparation comprising a plurality of doses for administration, each dose at least one aromatase inhibitor and an estrogen, and instructions for use in a male in need of hormone replacement therapy,

The present invention further provides a method of treating a male in need of hormone replacement therapy comprising administering to said male a pharmaceutical regimen comprising a plurality of doses, each dose comprising at least one aromatase inhibitor and an estrogen.

The present invention further provides the use of at least one aromatase inhibitor and a dose of estrogen in the preparation of a medicament, characterized in that the medicament is hormone replacement therapy for administration to a male in need of such therapy, the medicament comprising a plurality of doses for simultaneous, separate or sequential administration, each dose comprising at least one aromatase inhibitor and an estrogen.

In a further aspect, the invention provides the use of an aromatase inhibitor and an estrogen for the treatment of mood disorder, such as loss of libido and/or depression.

### DETAILED DESCRIPTION OF THE INVENTION

The present invention provides a pharmaceutical preparation comprising at least one aromatase inhibitor and estrogen, preferably a physiological replacement dose of estrogen, for administration to a male with androgen deficiency symptoms. The aromatase inhibitor will block aromatization of androgen to estrogen, resulting in reduced negative feedback on LH and FSH levels centrally. The dose of estrogen will be selected such that it is below the dose that would prevent the rise in gonadotropins. Increased LH stimulates testicular interstitial cell testosterone secretion, and increased FSH increases spermatogenesis. In addition, androstenedione and testosterone levels will also be elevated by prevention of peripheral aromatization of these substrates to estrogen in muscle, fat and other tissues, as described in Nelson L R. Et al., J Am Acad Dermatol 2001; 45:S116-24. Therefore a combination of central and peripheral effects increases endogenous androgen levels.

The increase in androgen, especially the increase in endogenous testosterone, improves muscle mass, reduces fat mass and reduces cardiovascular system risk. Since the present invention takes advantage of endogenous androgens, it avoids the side effects inherent in present methods of exogenous testosterone administration described above. The addition of a low dose of estrogen will act together with the androgen increase to improve libido, while preventing estrogen deficiency loss of bone mineral density and improving serum lipid profile and other potentially beneficial cardiovascular effects, described in Taxel P. et al., Endocr Res 2000; 26:381-98, and Lombardi G. et al., Mol Cell Endocrinol 2001; 178:51-5.

According to the method, the patient is administered an aromatase inhibitor and an estrogen. The estrogen is preferably administered in a physiologic replacement dose.

Also provided is the use of an aromatase inhibitor and an estrogen for the treatment of a mood disorder such as depression or loss of libido.

In all aspects of the invention, each dose of the aromatase inhibitor and each dose of the estrogen need not be administered simultaneously. Also within the scope of the invention are regimens in which the estrogen is administered alternately with the aromatase inhibitor at periodic intervals. For example, the aromatase inhibitor may be administered in the morning, and the estrogen may be administered in the evening, or vice versa. Also contemplated are regimens in which the aromatase inhibitor and the estrogen are administered on alternate days. It is also possible to administer a single dose of aromatase inhibitor (for example 10-30 mg of Anastrozole or the bio-equivalent dose of another aromatase inhibitor), every three, four or five days, together with an estrogen, either daily or every second, third or fourth day.

Definitions

Aromatase Inhibitor

By “aromatase inhibitors” there are to be understood substances that inhibit the enzyme aromatase (=oestrogen synthetase), which is responsible for converting androgens to oestrogens.

Aromatase inhibitors may have a -non-steroidal or a steroidal chemical structure. According to the present invention, both non-steroidal aromatase inhibitors and steroidal aromatase inhibitors can be used.

By aromatase inhibitors there are to be understood especially those substances that in a determination of the in vitro inhibition of aromatase activity exhibit IC₅₀ values of 10⁻⁵ M or lower, especially 10⁻⁶ M or lower, preferably 10⁻⁷ M or lower and most especially 10⁻⁸ M or lower.

The in vitro inhibition of aromatase activity can be demonstrated, for example, using the methods described in J. Biol. Chem. 249, 5364 (1974) or in J. Enzyme Inhib. 4, 169 (1990). In addition, IC₅₀ values for aromatase inhibition can be obtained, for example, in vitro by a direct product isolation method relating to inhibition of the conversion of 4-¹⁴C-androstenedione to 4-¹⁴C-oestrone in human placental microsomes.

By aromatase inhibitors there are to be understood most especially substances for which the minimum effective dose in the case of in vivo aromatase inhibition is 10 mg/kg or less, especially 1 mg/kg or less, preferably 0.1 mg/kg or less and most especially 0.01 mg/kg or less.

In vivo aromatase inhibition can be determined, for example, by the following method [see J. Enzyme Ihib. 4, 179 (1990)]: androstenedione (30 mg/kg subcutaneously) is administered on its own or together with a compound of the invention (orally or subcutaneously) to sexually immature female rats for a period of 4 days. After the fourth administration, the rats are sacrificed and the uteri are isolated and weighed. The aromatase inhibition is determined by the extent to which the hypertrophy of the uterus induced by the administration of androstenedione alone is suppressed or reduced by the simultaneous administration of the compound according to the invention.

The following groups of compounds are listed as examples of aromatase inhibitors. Each individual group forms a group of aromatase inhibitors that can be used successfully in accordance with the present invention:

The compounds of formulae I and I* as defined in EP-A-165 904. These are especially the compounds of formula I

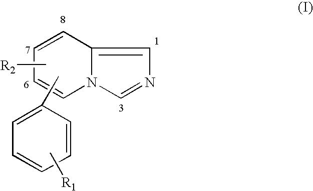

wherein R₁ is hydrogen, lower alkyl; lower alkyl substituted by hydroxy, lower alkoxy, lower alkanoyloxy, lower alkanoyl, amino, lower alkylamino, di-lower alkylamino, halogen, sulfo, carboxy, lower alkoxycarbonyl, carbamoyl or by cyano; nitro, halogen, hydroxy, lower alkoxy, lower alkanoyloxy, phenylsulfonyloxy, lower alkylsulfonyloxy, mercapto, lower alkylthio, lower alkylsulfinyl, lower alkylsulfonyl, lower alkanoylthio, amino, lower alkylamino, di-lower alkylamino, lower alkyleneamino, N-morpholino, N-thiomorpholino, N-piperazino that is unsubstituted or lower alkyl-substituted in the 4-position, tri-lower alkylammonio, sulfo, lower alkoxysulfonyl, sulfamoyl, lower alkylsulfamoyl, di-lower alkylsulfamoyl, formyl; iminomethyl that is unsubstituted or substituted at the nitrogen atom by hydroxy, lower alkoxy, lower alkanoyloxy, lower alkyl, phenyl or by amino; C₂-C₇ alkanoyl, benzoyl, carboxy, lower alkoxycarbonyl, carbamoyl, lower alkylcarbamoyl, di-lower alkylcarbamoyl, cyano, 5-tetrazolyl, unsubstituted or lower alkyl-substituted 4,5-dihydro-2-oxazolyl or hydroxycarbamoyl; and R₂ is hydrogen, lower alkyl, phenyl-lower alkyl, carboxy-lower alkyl, lower alkoxycarbonyl-lower alkyl, halogen, hydroxy, lower alkoxy, lower alkanoyloxy, mercapto, lower alkylthio, phenyl-lower alkylthio, phenylthio, lower alkanoylthio, carboxy, lower alkoxycarbonyl or lower alkanoyl; the 7,8-dihydro derivatives thereof; and the compounds of formula I*

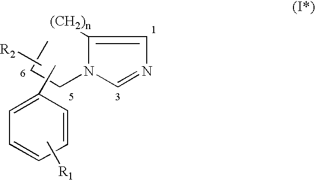

wherein n is 0, 1, 2, 3 or 4; and R₁ and R₂ are as defined above for formula I; it being possible for the phenyl ring in the radicals phenylsulfonyloxy, phenyliminomethyl, benzoyl, phenyl-lower alkyl, phenyl-lower alkylthio and phenylthio to be unsubstituted or substituted by lower alkyl, lower alkoxy or by halogen; it being possible in a compound of formula I* for the two substituents C₆H₄—R₁ and R₂ to be linked to each of the saturated carbon atoms of the saturated ring, either both to the same carbon atom or both to different carbon atoms, and pharmaceutically acceptable salts thereof.

Individual compounds that may be given special mention here are:

(1) 5-(p-cyanophenyl)imidazo[1,5-a]pyridine, 0

(2) 5-(p-ethoxycarbonylphenyl)imidazo[1,5-a]pyridine,

(3) 5-(p-carboxyphenyl )imidazo[1,5-a]pyridine,

(4) 5-(p-tert-butylaminocarbonylphenyl)imidazo[1,5-a]pyridine,

(5) 5-(p-ethoxycarbonylphenyl)-5,6,7,8-tetrahydroimidazo[1,5-a]pyridine,

(6) 5-(p-carboxyphenyl)-5,6,7,8-tetrahydroimidazo[1,5-a]pyridine,

(7) 5-(p-carbamoylphenyl)-5,6,7,8-tetrahydroimidazo[1,5-a]pyridine,

(8) 5-(p-tolyl)-5,6,7,8-tetrahydroimidazo[1,5-a]pyridine,

(9) 5-(p-hydroxymethylphenyl)imidazo[1,5-a]pyridine,

(10) 5-(p-cyanophenyl)-7,8-dihydroimidazo[1,5-a]pyridine,

(11) 5-(p-bromophenyl)-5,6,7,8-tetrahydroimidazo[1,5-a]pyridine,

(12) 5-(p-hydroxymethylphenyl)-5,6,7,8-tetrahydroimidazo[1,5-a]pyridine,

(13) 5-(p-formylphenyl)-5,6,7,8-tetrahydroimidazo[1,5-a]pyridine,

(14) 5-(p-cyanophenyl)-5-methylthio-5,6,7,8-tetrahydroimidazo[1,5-a]pyridine,

(15) 5-(p-cyanophenyl)-5-ethoxycarbonyl-5,6,7,8-tetrahydroimidazo[1,5-a]pyridine,

(16) 5-(p-aminophenyl)-5,6,7,8-tetrahydroimidazo[1,5-a]pyridine,

(17) 5-(p-formylphenyl)imidazo[1,5-a]pyridine,

(18) 5-(p-carbamoylphenyl)imidazo[1,5-a]pyridine,

(19) 5H-5-(4-tert-butylaminocarbonylphenyl)-6,7-dihydropyrrolo[1,2-c]imidazole,

(20) 5H-5-(4-cyanophenyl)-6,7-dihydropyrrolo[1,2-c]imidazole,

(21) 5H-5-(4-cyanophenyl)-6,7,8,9-tetrahydroimidazo[1,5-a]azepine,

(22) 5-(4-cyanophenyl)-6-ethoxycarbonylmethyl-5,6,7,8-tetrahydroimidazo[1,5-a]pyridine,

(23) 5-(4-cyanophenyl)-6-carboxymethyl-5,6,7,8-tetrahydroimidazo[1,5-a]pyridine

(24) 5-benzyl-5-(4-cyanophenyl)-5,6,7,8-tetrahydroimidazo[1,5-a]pyridine,

(25) 7-(p-cyanophenyl)-5,6,7,8-tetrahydroimidazo[1,5-a]pyridine,

(26) 7-(p-carbamoylphenyl)-5,6,7,8-tetrahydroimidazo[1,5-a]pyridine,

(27) 5-(p-cyanophenyl)-5,6,7,8-tetrahydroimidazo[1,5-a]pyridine (=Fadrozol).

The compounds of formula I as defined in EP-A 236 940. These are especially the compounds of formula I

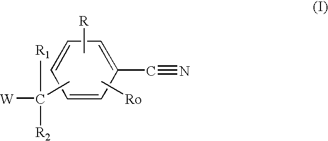

wherein R and R₀, independently of one another, are each hydrogen or lower alkyl, or R and R₀ at adjacent carbon atoms, together with the benzene ting to which they are bonded, form a naphthalene or tetrahydronaphthalene ring; wherein R₁ is hydrogen, lower alkyl, aryl, aryl-lower alkyl or lower alkenyl; R₂ is hydrogen, lower alkyl, aryl, aryl-lower alkyl, (lower alkyl, aryl or aryl-lower alkyl)-thio or lower alkenyl, or wherein R₁ and R₂ together are lower alkylidene or C₄-C₆ alkylene; wherein W is 1-imidazolyl, 1-(1,2,4 or 1,3,4)-triazolyl, 3-pyridyl or one of the mentioned heterocyclic radicals substituted by lower alkyl; and aryl within the context of the above definitions has the following meanings: phenyl that is unsubstituted or substituted by one or two substituents from the group lower alkyl, lower alkoxy, hydroxy, lower alkanoyloxy, nitro, amino, halogen, trifluoromethyl, cyano, carboxy, lower alkoxycarbonyl, carbamoyl, N-lower alkylcarbamoyl, N,N-di-lower alkylcarbamoyl, lower alkanoyl, benzoyl, lower alkylsulfonyl, sulfamoyl, N-lower alkylsulfamoyl and N,N-di-lower alkylsulfamoyl; also thienyl, indolyl, pyridyl or furyl, or one of the four last-mentioned heterocyclic radicals monosubstituted by lower alkyl, lower alkoxy, cyano or by halogen; and pharmaceutically acceptable salts thereof.

Individual compounds from that group that may be given special mention are:

(1) 4-[alpha-(4-cyanophenyl)-1-imidazolylmethyl]-benzonitrile,

(2) 4-[alpha-(3-pyridyl )-1-imidazolylmethyl]-benzonitrile,

(3) 4-[alpha-(4-cyanobenzyl)-1-imidazolylmethyl]-benzonitrile,

(4) 1-(4-cyanophenyl)-1-(1-imidazolyl)-ethylene,

(5) 4-[alpha-(4-cyanophenyl)-1-(1,2,4-triazolyl)methyl]-benzonitrile,

(6) 4-[alpha-(4-cyanophenyl)-3-pyridylmethyl]-benzonitrile.

The compounds of formula I as defined in EP-A-408 509. These are especially the compounds of formula I

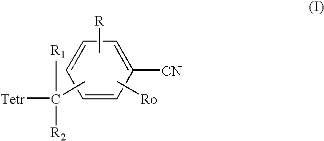

wherein Tetr is 1- or 2-tetrazolyl that is unsubstituted or substituted in the 5-position by lower alkyl, phenyl-lower alkyl or by lower alkanoyl; R and R₂, independently of one another, are each hydrogen; lower alkyl that is unsubstituted or substituted by hydroxy, lower alkoxy, halogen, carboxy, lower alkoxycarbonyl, (amino, lower alkylamino or di-lower alkylamino)-carbonyl or by cyano; lower alkenyl, aryl, heteroaryl, aryl-lower alkyl, C₃-C₆ cycloalkyl, C₃-C₆ cycloalkyl-lower alkyl, lower alkylthio, arylthio or aryl-lower alkylthio; or R₁ and R₂ together are straight-chained C₄-C₆ alkylene that is unsubstituted or substituted by lower alkyl, or are a group —(CH₂)ₘ-1,2-phenylene-(CH₂)ₙ— wherein m and n, independently of one another, are each 1 or 2 and 1,2-phenylene is unsubstituted or substituted in the same way as phenyl in the definition of aryl below, or are lower alkylidene that is unsubstituted or mono- or di-substituted by aryl; and R and R₀, independently of one another, are each hydrogen or lower alkyl; or R and R₀ together, located at adjacent carbon atoms of the benzene ring, are a benzo group that is unsubstituted or substituted in the same way as phenyl in the definition of aryl below; aryl in the above definitions being phenyl that is unsubstituted or substituted by one or more substituents from the group consisting of lower alkyl, lower alkoxy, hydroxy, lower alkanoyloxy, nitro, amino, halogen, trifluoromethyl, carboxy, lower alkoxycarbonyl, (amino, lower alkylamino or di-lower alkylamino)-carbonyl, cyano, lower alkanoyl, benzoyl, lower alkylsulfonyl and (amino, lower alkylamino or di-lower alkylamino)-sulfonyl; heteroaryl in the above definitions being an aromatic heterocyclic radical from the group consisting of pyrrolyl, pyrazolyl, imidazolyl, triazolyl, tetrazolyl, furanyl, thienyl, isoxazolyl, oxazolyl, oxadiazolyl, isothiazolyl, thiazolyl, thiadiazolyl, pyridyl, pyridazinyl, pyrimidyl, pyrazinyl, triazinyl, indolyl, isoindolyl, benzimidazolyl, benzotriazolyl, benzofuranyl, benzothienyl, benzoxazolyl, benzothiazolyl, benzoxadiazolyl, benzothiadiazolyl, quinolyl and isoquinolyl that is unsubstituted or substituted in the same way as phenyl in the definition of aryl above; and pharmaceutically acceptable salts thereof.

Individual compounds from that group that may be given special mention are:

(1) 4-(2-tetrazolyl)methyl-benzonitrile,

(2) 4-[α-(4-cyanophenyl )-(2-tetrazolyl )methyl]-benzonitrile,

(3) 1-cyano-4-(1-tetrazolyl)methyl-naphthalene,

(4) 4-[α-(4-cyanophenyl)-(1-tetrazolyl)methyl]-benzonitrile.

The compounds of formula I as defined in European Patent Application No. 91810110.6. These are especially the compounds of formula I

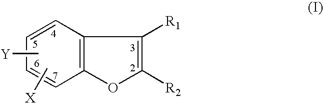

wherein X is halogen, cyano, carbamoyl, N-lower alkylcarbamoyl, N-cycloalkyl-lower alkylcarbamoyl, N,N-di-lower alkylcarbamoyl, N-arylcarbamoyl, hydroxy, lower alkoxy, aryl-lower alkoxy or aryloxy, wherein aryl is phenyl or naphthyl, each of which is unsubstituted or substituted by lower alkyl, hydroxy, lower alkoxy, halogen and/or by trifluoromethyl; Y is a group —CH₂A wherein A is 1-imidazolyl, 1-(1,2,4-triazolyl), 1-(1,3,4-triazolyl), 1-(1,2,3-triazolyl), 1-(1,2,5-triazolyl), 1-tetrazolyl or 2-tetrazolyl, or Y is hydrogen, R₁ and R₁, independently of one another, are each hydrogen, lower alkyl or a group —CH₂-A as defined for Y, or R₁ and R₂ together are —(CH₂)ₙ— wherein n is 3, 4 or 5, with the proviso that one of the radicals Y, R₁ and R₂ is a group —CH₂-A, with the further proviso that in a group —CH₂-A as a meaning of R₁ or R₂, A is other than 1-imidazolyl when X is bromine, cyano or carbamoyl, and with the proviso that in a group —CH.₂-A as a meaning of Y, A is other than 1-imidazolyl when X is halogen or lower alkoxy, R₁ is hydrogen and R₂ is hydrogen or lower alkyl, and pharmaceutically acceptable salts thereof.

Individual compounds from that group that may be given special mention are:

(1) 7-cyano-4-[l-(1,2,4-triazolyl)methyl]-2,3-dimethylbenzofuran,

(2) 7-cyano-4-(1-imidazolylmethyl)-2,3-dimethylbenzofuran,

(3) 7-carbamoyl-4-(1-imidazolylmethyl)-2,3-dimethylbenzofuran,

(4) 7-N-(cyclohexylmethyl)carbamoyl-4-(1-imidazolylmethyl)-2,3-dimethylbenzofuran.

The compounds of formula I as defined in Swiss Patent Application 1339/90-7.

These are especially the compounds of formula I

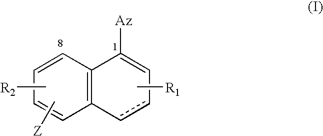

wherein the dotted line denotes an additional bond or no additional bond, Az is imidazolyl, triazolyl or tetrazolyl bonded via a ring nitrogen atom, each of those radicals being unsubstituted or substituted at carbon atoms by lower alkyl or by aryl-lower alkyl, Z is carboxy, lower alkoxycarbonyl, carbamoyl, N-lower alkylcarbarmoyl, N,N-di-lower alkylcarbamoyl, N-arylcarbamoyl, cyano, halogen, hydroxy, lower alkoxy, aryl-lower alkoxy, aryloxy, lower alkyl, trifluoromethyl or aryl-lower alkyl, and R₁ and R₂, independently of one another, are each hydrogen, lower alkyl, lower alkoxy, hydroxy, halogen or trifluoromethyl; aryl being phenyl or naphthyl each of which is unsubstituted or substituted by one or two substituents from the group consisting of lower alkyl, lower alkoxy, hydroxy, halogen and trifluoromethyl; with the proviso that neither Z nor R₂ is hydroxy in the 8-position, and pharmaceutically acceptable salts thereof.

Individual compounds from that group that may be given special mention are:

(1) 6-cyano-1-(1-imidazolyl)-3,4-dihydronaphthalene,

(2) 6-cyano-1-[1-(1,2,4-triazolyl)]-3,4-dihydronaphthalene,

(3) 6-chloro-1-(1-imidazolyl)-3,4-dihydronaphthalene,

(4) 6-bromo-1-(1-imidazolyl)-3,4-dihydronaphthalene.

The compounds of formula I as defined in Swiss Patent Application 3014/90-0.

These are especially the compounds of formula I

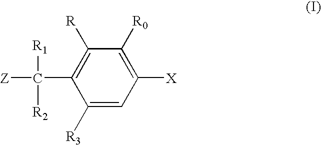

wherein Z is a five-membered nitrogen-containing heteroaromatic ting selected from the group 5-isothiazolyl, 5-thiazolyl, 5-isoxazolyl, 5-oxazolyl, 5-(1,2,3-thiadiazolyl), 5-(1,2,3-oxadiazolyl), 3-(1,2,5-thiadiazolyl), 3-(1,2,5-oxadiazolyl), 4-isothiazolyl, 4-isoxazolyl, 4-(1,2,3-thiadiazolyl), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl); R and R₀ are hydrogen; or R and R₀ together are a benzo group that is unsubstituted or substituted by lower alkyl, lower alkoxy, hydroxy, halogen or by trifluoromethyl; R₁ is hydrogen, hydroxy, chlorine or fluorine; R₃ is hydrogen; R₂ is hydrogen, lower alkyl or phenyl that is unsubstituted or substituted by lower alkyl, lower alkoxy, hydroxy, halogen, trifluoromethyl or by cyano; or R₁ and R₂ together are methylidene; or R₂ and R₃ together are —(CH₂)₃—; or R₁ and R₂ and R₃ together are a group ═CH—(CH₂)₂— wherein the single bone is linked to the benzene ring; X is cyano; and X may also be halogen when R₂ and R₃ together are —(CH₂)₃— or R₁ and R₂ and R₃ together are a group ═CH—(CH₂)₂—; and pharmaceutically acceptable salts thereof.

Individual compounds from that group that may be given special mention are:

(1) 4-[α-(4-cyanophenyl)-α-hydroxy-5-isothiazolylmethyl]-benzonitrile.

(2) 4-[α-(4-cyanophenyl)-5-isothiazolylmethyl]-benzonitrile,

(3) 4-[α-(4-cyanophenyl)-5-thiazolylmethyl]-benzonitrile,

(4) 1-(4-cyanophenyl)-1-(5-tiazolyl)-ethylene,

(5) 6-cyano-1-(S-isothiazolyl )-3,4-dihydronaphthalene,

(6) 6-cyano-1-(5-thiazolyl)-3,4-dihydronaphthalene.

The compounds of formula VI as defined in Swiss Patent Application 3014/90-0.

These are especially the compounds of formula VI

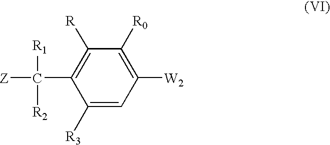

wherein Z is a five-membered nitrogen-containing heteroaromatic ring selected from the group 5-isothiazolyl, 5-thiazolyl, 5-isoxazolyl, 5-oxazolyl, 5-(1,2,3-thiadiazolyl). 5-(1,2,3-oxadiazolyl) 3-(1,2,5-thiadiazolyl), 3-(1,2,5-oxadiazolyl), 4-isothiazolyl. 4-isoxazolyl, 4-(1,2,3-thiadiazolyl), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl); R and R₀ are each hydrogen; or R and R₀ together are a benzo group that is unsubstituted or substituted by lower alkyl, lower alkoxy, hydroxy, halogen or by trifluoromethyl; R₁ is hydrogen, hydroxy, chlorine or fluorine; R₃ is hydrogen; R₂ is hydrogen, lower alkyl or phenyl that is unsubstituted or substituted by lower alkyl, lower alkoxy, hydroxy, halogen, trifluoromethyl, aryl-lower alkoxy or by aryloxy; or R₁ and R₂ together are methylidene, and W₂ is halogen, hydroxy, lower alkoxy, aryl-lower alkoxy or aryloxy; aryl in each case being phenyl that is unsubstituted or substituted by lower alkyl, lower alkoxy, hydroxy, halogen or by trifluoromethyl; and pharmaceutically acceptable salts thereof.

Individual compounds from that group that may be given special mention are:

bis(4,4′-bromophenyl)-(5-isothiazolyl)methanol,

bis(4,4′-bromophenyl)-(5-isothiazolyl)methane,

bis(4,4′-bromophenyl)-(5-thiazolyl)methanol,

bis(4,4′-bromophenyl)-(5-thiazolyl)methane.

The compounds of formula I as defined in Swiss Patent Application 3923/90-4.

These are especially the compounds of formula I

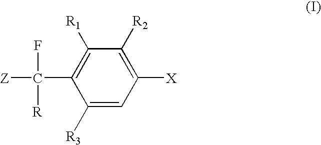

wherein Z is imidazolyl, triazolyl, tetrazolyl, pyrrolyl, pyrazolyl, indolyl, isoindolyl, benzinidazolyl, benzopyrazolyl, benzotriazolyl, pyndyl, pyrimidyl, pyrazinyl, pyridazinyl. triazinyl, quinolinyl or isoquinolinyl, all those radicals being bonded via their heterocyclic rings and all those radicals being unsubstituted or substituted by lower alkyl, hydroxy, lower alkoxy, halogen or by trifluoromethyl: R₁ and R₂, independently of one another, are each hydrogen or lower alkyl; or R₁ and R₂ together are C₃-C₄ alkylene, or a benzo group that is unsubstituted or substituted as indicated below for aryl; R is hydrogen, lower alkyl, aryl or heteroaryl, and X is cyano, carbamoyl, N-lower alkylcarbamoyl, N,N-di-lower alkylcarbamoyl, N,N-lower alkylenecarbamoyl; N,N-lower alkylenecarbamoyl interrupted by —O—, —S— or —NR″—, wherein R″ is hydrogen, lower alkyl or lower alkanoyl; N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-aryl-lower alkylcarbamoyl, N-arylcarbamoyl, N-hydroxycarbamoyl, hydroxy, lower alkoxy, aryl-lower alkoxy or aryloxy; and wherein X is also halogen when Z is imidazolyl, triazolyl, tetrazolyl, pyrrolyl, pyrazolyl, indolyl, isoindolyl, benzimidazolyl, benzopyrazolyl or benzotriazolyl; wherein aryl is phenyl or naphthyl, these radicals being unsubstituted or substituted by from 1 to 4 substituents from the group consisting of lower alkyl, lower alkenyl, lower alkynyl, lower alkylene (linked to two adjacent carbon atoms), C₃-C₈ cycloalkyl, phenyl-lower alkyl, phenyl; lower alkyl that is substituted in turn by hydroxy, lower alkoxy, phenyl-lower alkoxy, lower alkanoyloxy, halogen, amino, lower alkylamino, di-lower alkylamino, mercapto, lower alkylthio, lower alkylsulfinyl, lower alkylsulfonyl, carboxy, lower alkoxycarbonyl, carbamoyl, N-lower alkylcarbamoyl, N,N-di-lower alkylcarbamoyl and/or by cyano; hydroxy; lower alkoxy, halo-lower alkoxy, phenyl-lower alkoxy, phenoxy, lower alkenyloxy, halo-lower alkenyloxy, lower alkynyloxy, lower alkylenedioxy (linked to two adjacent carbon atoms), lower alkanoyloxy, phenyl-lower alkanoyloxy, phenylcarbonyloxy, mercapto, lower alkylthio, phenyl-lower alkylthio, phenylthio, lower alkylsulfinyl, phenyl-lower alkylsulfinyl, phenylsulfinyl, lower alkylsulfonyl, phenyl-lower alkylsulfonyl, phenylsulfonyl, halogen, nitro, amino, lower alkylamino, C₃-C₈ cycloalkylamino, phenyl-lower alkylamino, phenylamino, di-lower alkylamino, N-lower alkyl-N-phenylamino, N-lower alkyl-N-phenyl-lower alkylamino; lower alkyleneamino or lower alkyleneamino interrupted by —O—, —S— or —NR″— (wherein R″ is hydrogen, lower alkyl or lower alkanoyl); lower alkanoylamino, phenyl-lower alkanoylamino, phenylcarbonylamino, lower alkanoyl, phenyl-lower alkanoyl, phenylcarbonyl, carboxy, lower alkoxycarbonyl, carbamoyl, N-lower alkylcarbamoyl, N,N-di-lower alkylcarbamoyl, N,N-lower alkylenecarbamoyl; N,N-lower alkylenecarbamoyl interrupted by —O—, —S— or —NR″—, wherein R″ is hydrogen, lower alkyl or lower alkanoyl; N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-hydroxycarbamoyl, N-phenyl-lower alkylcarbamoyl, N-phenylcarbamoyl, cyano, sulfo, lower alkoxysulfonyl, sulfamoyl, N-lower alkylsulfamoyl, N,N-di-lower alkylsulfamoyl and N-phenylsulfamoyl; the phenyl groups occurring in the substituents of phenyl and naphthyl in turn being unsubstituted or substituted by lower alkyl, lower alkoxy, hydroxy, halogen and/or by trifluoromethyl; wherein heteroaryl is indolyl, isoindolyl, benzimidazolyl, benzopyrazolyl, benzotriazolyl, benzo[b]furanyl, benzo[b]thienyl, benzoxazolyl or benzothiazolyl, those radicals being unsubstituted or substituted by from 1 to 3 identical or different substituents selected from lower alkyl, hydroxy, lower alkoxy, halogen, cyano and trifluoromethyl; and pharmaceutically acceptable salts thereof.

Those compounds are especially the compounds of formula I whereto Z is 1-imidazolyl, 1-(1,2,4-triazolyl), 1-(1,3,4-triazolyl), 1-(1,2,3-triazolyl), 1-tetrazolyl, 2-tetrazolyl, 3-pyridyl, 4-pyrimidyl, 5-pyrimidinyl or 2-pyrazinyl; R₁ and R₂, independently of one another, are each hydrogen or lower alkyl; or R₁ and R₂ together are 1,4-butylene or a benzo group; R is lower alkyl; phenyl that is unsubstituted or substituted by cyano, carbamoyl, halogen, lower alkyl, trifluoromethyl, hydroxy, lower alkoxy or by phenoxy; or benzotriazolyl or benzo[b]furanyl, the last two radicals being unsubstituted or substituted by from 1 to 3 identical or different substituents selected from lower alkyl, halogen and cyano; and X is cyano or carbamoyl; and wherein X is also halogen when Z is 1-imidazolyl, 1-(1,2,4-triazolyl), 1-(1,3,4-triazolyl), 1-(1,2,3-triazolyl), 1-tetrazolyl 2-tetrazolyl; and pharmaceutically acceptable salts thereof.

Individual compounds that may be given special mention here are:

(1) 4-[α-4-cyanophenyl)-α-fluoro-1-(1,2,4-triazolyl)methyl]-benzonitrile,

(2) 4-[α-(4-cyanophenyl)-α-fluoro-(2-tetrazolyl)methyl]-benzonitrile,

(3) 4-[α-(4-cyanophenyl)-α-fluoro-(1-tetrazolyl)methyl]-benzonitrile,

(4) 4-[α-(4-cyanophenyl)-α-fluoro-(1-imidazolyl)methyl]-benzonitrile,

(5) 1-methyl-6-[α-(4-chlorophenyl)-α-fluoro-1-(1,2,4-triazolyl)methyl]-benzotriazole,

(6) 4-[α-(4-cyanophenyl)-α-fluoro-1-(1,2,3-triazolyl)methyl]-benzonitrile,

(7) 7-cyano-4-[α-(4-cyanophenyl)-α-fluoro-1-(1,2,4-triazolyl)methyl]-2,3-dimethylbenzo[b]furan,

(8) 4-[α-(4-bromophenyl)-α-fluoro-1-(1,2,4-triazolyl)methyl]-benzonitrile,

(9) 4-[α-(4-cyanophenyl)-α-fluoro-(5-pyrimidyl)methyl]-benzonitrile,

(10) 4-[α-(4-bromophenyl)-α-fluoro-(5-pyrimidyl)methyl]-benzonitrile,

(11) 4-[α-(4-cyanophenyl)-α-fluoro-(3-pyridyl)methyl]-benzonitrile,

(12) 7-bromo4-[α-(4-cyanophenyl)-α-fluoro-(1-imidazolyl)methyl]-2,3-dimethylbenzo[b]furan,

(13) 7-bromo-4-[α-(4-cyanophenyl)-α-fluoro-1-(1,2,4-triazolyl)methyl]-2,3-dimethylbenzo[b]furan,

(14) 4-[α-(4-cyanophenyl)-α-fluoro-(5-pyrimidyl)methyl]-benzonitrile,

(15) 4-[α-(4-bromophenyl)-α-fluoro-(5-pyrimidyl)methyl]-benzonitrile,

(16) 4-[α-(4-cyanophenyl)-1-(1,2,3-triazolyl)methyl]-benzonitrile,

(17) 2,3-dimethyl4-[α-(4-cyanophenyl)-1-(1,2,4-triazolyl)methyl]-7-cyano-benzo[b]furan,

(18) 4-[α-(4-cyanophenyl)-(5-pyrimidyl)methyl]-benzonitrile,

(19) 4-[α-(4-bromophenyl)-(5-pyrimidyl)methyl]-benzonitrile,

(20) 2,3-dimethyl4-[α-(4-cyanophenyl)-(1-imidazolyl)methyl]-7-bromo-benzo[b]furan,

(21) 2,3-dimethyl-4-[α-(4-cyanophenyl)-1-(1,2,4-triazolyl)methyl]-7-bromo-benzo-[b]furan.

The compounds of formula I as defined in EP-A-114 033. These are especially the compounds of formula I

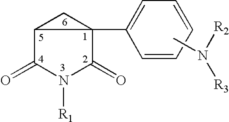

wherein R₁ is hydrogen, R₂ is hydrogen, sulfo, C₁-C₇ alkanoyl or C₁-C₇ alkanesulfonyl and R₃ is hydrogen, or wherein R₁ is C₁-C₁₂ alkyl, C.₂-C₁₂ alkenyl, C₂-C₇ alkynyl, C₃-C₁₀ cycloalkyl, C₃-C.₁₀ cycloalkenyl, C.₃3-C₆ cycloalkyl-C₁-C₄ alkyl, C₃-C₆ cycloalkyl-C.₂-C₄ alkenyl or C₃-C₆ cycloalkenyl-C₁-C₄ alkyl, R₂ is hydrogen, C₁-C₇ alkyl, sulfo, C₁-C₇ alkanoyl or C₁-C₇ alkanesulfonyl and R₃ is hydrogen or C₁-C₇ alkyl, and salts of those compounds.

Individual compounds from that group that may be given special mention are:

(1) 1-(4-aminophenyl)-3-methyl-3-azabicyclo[3.1.0]hexane-2,4-dione,

(2) 1-(4-aminophenyl)-3-n-propyl-3-azabicyclo[3.1.0]hexane-2,4-dione,

(3) 1-(4-aminophenyl)-3-isobutyl-3-azabicyclo[3.1.0]hexane-2,4-dione,

(4) 1-(4-aminophenyl)-3-n-heptyl-3-azabicyclo[3.1.0]hexane-2,4-dione,

(5) 1-(4-aminophenyl)-3-cyclohexylmethyl-3-azabicyclo[3.1.0]hexane-2,4-dione.

The compounds of formula I as defined in EP-A-166 692. These are especially the compounds of formula I

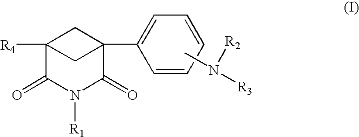

wherein R₁ is hydrogen, alkyl having from 1 to 12 carbon atoms, alkenyl having from 2 to 12 carbon atoms, lower alkynyl, cycloalkyl or cycloalkenyl each having from 3 to 10 carbon atoms, cycloalkyl-lower alkyl having from 4 to 10 carbon atoms, cycloalkyl-lower alkenyl having from 5 to 10 carbon atoms, cycloalkenyl-lower alkyl having from 4 to 10 carbon atoms, or aryl having from 6 to 12 carbon atoms or aryl-lower alkyl having from 7 to 15 carbon atoms, each of which is unsubstituted or substituted by lower alkyl, hydroxy, lower alkoxy, acyloxy, amino, lower alkylamino, di-lower alkylamino, acylamino amino or by halogen, R₂ is hydrogen, lower alkyl, sulfo, lower alkanoyl or lower alkanesulfonyl, sulfonyl, R₃ is hydrogen or lower alkyl and R₄ is hydrogen, lower alkyl, phenyl or phenyl substituted by —N(R₂)(R₃), and salts thereof, radicals described as “lower” containing up to and including 7 carbon atoms.

Individual compounds from that group that may be given special mention are:

(1) 1-(4-aminophenyl)-3-n-propyl-3-azabicyclo[3.1.1]heptane-2,4-dione,

(2) 1-(4-aminophenyl)-3-methyl-3-azabicyclo[3.1.1]heptane-2,4-dione,

(3) 1-(4-aminophenyl)-3-n-decyl-3-azabicyclo[3.1.1]heptane-2,4-dione,

(4) 1-(4-aminophenyl)-3-cyclohexyl-3-azabicyclo[3.1.1]heptane-2,4-dione,

(5) 1-(4-aminophenyl)-3-cyclohexylmethyl-3-azabicyclo[3.1.1]heptane-2,4-dione.

The compounds of formula I as defined in EP-A-356 673. These are especially the compounds of formula I

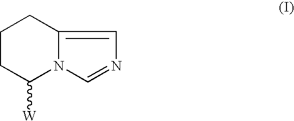

wherein W (α) is a 2-naphthyl or 1-anthryl radical, wherein each benzene ring is unsubstituted or substituted by a substituent selected from halogen, hydroxy, carboxy, cyano and nitro; or (.beta.) is 4-pyridyl, 2-pyrimidyl or 2-pyrazinyl, each of those radicals being unsubstituted or substituted by a substituent selected from halogen, cyano, nitro, C₁-C₄ alkoxy and C₂-C₅ alkoxycarbonyl; and pharmaceutically acceptable salts thereof.

Individual compounds from that group that may be given special mention are:

(1) 5-(2′-naphthyl)-5,6,7,8-tetrahydroimidazo[1,5-a]pyridine,

(2) 5-(4′-pyridyl)-5,6,7,8-tetrahydroimidazo[1,5-a]pyridine.

The compounds of formula I or Ia as defined in EP-A-337 929. These are especially the compounds of formula I/Ia

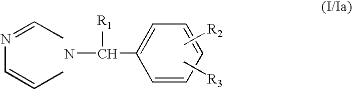

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, R₂ is benzyloxy, 3-bromo-, 4-bromo-, 4-chloro-, 2,3-, 2,4-, 4,5- or 4,6-dichloro-benzyloxy, and R₃ is cyano; C₂-C₁₀ alkanoyl that is unsubstituted or mono- or poly-substituted by halogen, methoxy, amino, hydroxy and/or by cyano; benzoyl that is unsubstituted or substituted by one or more substituents from the group halogen, C₁-C₄ alkyl, methoxy, amino, hydroxy and cyano; carboxy, (methoxy, ethoxy or butoxy)-carbonyl, carbamoyl, N-isopropylcarbamoyl, N-phenylcarbamoyl, N-pyrrolidylcarbonyl, nitro or amino; and salts thereof.

Individual compounds from that group that may be given special mention are:

(1) 4-(2,4-dichlorobenzyloxy)-3-[1-(1-imidazolyl)-butyl]-benzonitrile,

(2) (4-(4-bromobenzyloxy)-3-[1-(1-imidazolyl )-butyl]-phenyl pentyl ketone,

(3) 4-(4-bromobenzyloxy)-3-[1-(1-imidazolyl)-butyl]-benzanilide,

(4) 4-(4-bromobenzyloxy)-3-[1-(1-imidazolyl)-butyl]-benzoic acid,

(5) 3-(2,4-dichlorobenzyloxy)-4-[1-(1-imidazolyl)-butyl]-benzonitrile,

(6) 3-(2,4-dichlorobenzyloxy)-4-[1-(1-imidazolyl)-butyl]-benzoic acid methyl ester,

(7) 3-(2,4-dichlorobenzyloxy)-4-[1-(1-imidazolyl)-butyl]-benzoic acid,

(8) 3-(3-bromobenzyloxy)-4-[1-(1-imidazolyl )-butyl]-benzonitrile,

(9) 4-(3-bromobenzyloxy)-3-[1-(1-imidazolyl)-butyl]-benzonitrile,

(10) 3-(4-bromobenzyloxy)-4-[1-(1-imidazolyl)-butyl]-benzoic acid,

(11) 3-(4-bromobenzyloxy)-4-[1-(1-imidazolyl)-butyl]-benzanilide,

(12) 3-(4-bromobenzyloxy)-4-[1-(1-imidazolyl)-butyl]-phenyl pentyl ketone,

(13) 4-(4-bromobenzyloxy)-3-[1-(1-imidazolyl)-butyl]-benzonitrile,

(14) 3-(4-bromobenzyloxy)-4-[1-(1-imidazolyl)-butyl]-benzonitrile,

(15) 4-nitro-2-[1-(1-imidazolyl)-butyl]-phenyl-(2,4-dichlorobenzyl)ether,

(16) 4-amino-2-[1-(1-imidazolyl)-butyl]-phenyl-(2,4-dichlorobenzyl)ether,

(17) (2,4-dichlorobenzyl)-[2-(1-imidazolyl-methyl)-4-nitrophenyl]ether.

The compounds of formula I as defined in EP-A-337 928. These are especially the compounds of formula I

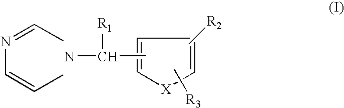

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, R₂ is hydrogen, halogen, cyano, methyl, hydroxymethyl, cyanomethyl, methoxymethyl, pyrrolidinylmethyl, carboxy, (methoxy, ethoxy or butoxy)-carbonyl, carbamoyl, N-isopropylcarbamoyl, N-phenylcarbamoyl, N-pyrrolidylcarbonyl; C₂-C₁₀ alkanoyl that is unsubstituted or mono- or poly-substituted by halogen, methoxy, ethoxy, amino, hydroxy and/or by cyano; or benzoyl that is unsubstituted or substituted by one or more substituents from the group halogen, C₁-C₄ alkyl, methoxy, ethoxy, amino, hydroxy and cyano, R₃ is hydrogen, benzyloxy, 3-bromo-, 4-bromo-, 4-chloro-, 2,3-, 2,4-, 4,5- or 4,6-dichlorobenzyloxy, and X is —CH═N—; —CH═N(—O)— or —S—; and salts thereof.

Individual compounds from that group that may be given special mention are:

(1) 5-[1-(1-imidazolyl)-butyl]-thiophene-2-carbonitrile,

(2) 2-[1-(1-imidazolyl)-butyl]-thiophene-4-carbonitrile,

(3) 2-[1-(1-imidazolyl)-butyl]-4-bromo-thiophene,

(4) 2-[1-(1-imidazolyl)-butyl]-5-bromo-thiophene,

(5) 5-[1-(1-imidazolyl)-butyl]-2-thienyl pentyl ketone,

(6) 5-[1-(1-imidazolyl)-butyl]-2-thienyl ethyl ketone,

(7) 5-(4-chlorobenzyloxy)-4-[1-(1-imidazolyl)-pentyl]-pyridine-2-carbonitrile,

(8) 3-(4-chlorobenzyloxy)-4-[1-(1-imidazolyl)-pentyl]-pyridine-2-carbonitrile,

(9) 3-(4-chlorobenzyloxy)-4-[1-(1-imidazolyl)-pentyl]-pyridine-N-oxide,

(10) 3-(4-chlorobenzyloxy)-4-[1-(1-imidazolyl)-pentyl]-pyridine.

The compounds of formula I as defined in EP-A-340 153. These are especially the compounds of formula I

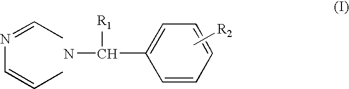

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, and R₂ is a radical from the group methyl, ethyl, propyl, benzyl, phenyl and ethenyl that is substituted by hydroxy, cyano, methoxy, butoxy, phenoxy, amino, pyrrolidinyl, carboxy, lower alkoxycarbonyl or by carbamoyl; or R₂ is formyl or derivatised formyl that can be obtained by reaction of the formyl group with an amine or amine derivative from the group hydroxylamine, O-methylhydroxylamine, O-ethylhydroxylamine, O-allylhydroxylamine, O-benzylhydroxylamine, O-4-nitrobenzyloxyhydroxylamine, O-2,3,4,5,6-pentafluorobenzyloxyhydroxylamine, semicarbazide, thiosemicarbazide, ethylamine and aniline; acetyl, propionyl, butryl, valeryl, caproyl; benzoyl that is unsubstituted or substituted by one or more substituents from the group halogen, C₁-C₄-alkyl, methoxy, amino, hydroxy and cyano; carboxy, (methoxy, ethoxy or butoxy)carbonyl, carbamoyl, N-isopropylcarbamoyl, N-phenylcarbamoyl or N-pyrrolidylcarbonyl; and salts thereof.

Individual compounds from that group that may be given special mention are:

(1) 4-(1-(1-imidazolyl)-butyl)-benzoic acid methyl ester,

(2) 4-(1-(1-imidazolyl)-butyl)-benzoic acid butyl ester,

(3) 4-(1-(1-imidazolyl)-butyl)-phenyl-acetonitrile,

(4) 4-(1-(1-imidazolyl)-butyl)-benzaldehyde,

(5) 4-(1-(1-imidazolyl)-butyl)-benzyl alcohol,

{4-[1-(1-imidazolyl)-butyl]-phenyl }-2-propyl ketone,

(7) 4-[1-(1-imidazolyl)-butyl]-phenyl propyl ketone,

(8) 4-[1-(1-imidazolyl)-butyl]-phenyl butyl ketone,

(9) 4-[1-(1-imidazolyl)-butyl]-phenyl pentyl ketone,

(10) 4-[1-(1-imidazolyl)-butyl]-phenyl hexyl ketone.

The compounds of formula I as defined in DE-A-4 014 006. These are especially the compounds of formula I

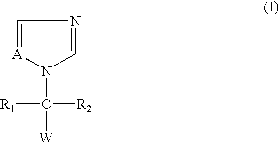

wherein A is an N-atom or a CH radical and W is a radical of the formula

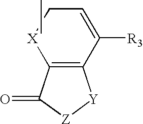

wherein X is an oxygen or a sulfur atom or a —CH═CH— group and Y is a methylene group, an oxygen or a sulfur atom and Z is a —(CH₂)ₙ— group wherein n=1, 2 or 3 and either

R₃ in W is a hydrogen atom and R₁ and R₂, independently of one another, are each a hydrogen atom, a C₁— to C₁₀ alkyl group or a C₃— to C₇ cycloalkyl group, or

R₂ is as defined under a) and R₁ together with R₃ forms a —(CH₂)ₘ— group wherein m=2, 3, or 4, and their pharmaceutically acceptable addition salts with acids.

Individual compounds from that group that may be given special mention are:

(1) 5-[1-(1-imidazolyl)-butyl]-1-indanone,

(2) 7-[1-(1-imidazolyl)-butyl]-1-indanone,

(3) 6-[1-(1-imidazolyl)-butyl]-1-indanone,

(4) 6-(1-imidazolyl)-6,7,8,9-tetrahydro-1H-benz[e]inden-3(2H)-one,

(5) 2-[1-(1-imidazolyl)-butyl]-4,-dihydro-6-oxo-cyclopenta[b]-thiophene,

(6) 6-[1-(1-imidazolyl)-butyl]-3,4-dihydro-2H-naphthalen-1-one,

(7) 2-[1-(1-imidazolyl)-butyl]-6,7-dihydro-5H-benzo[b]thiophen-4-one,

(8) 6-[1-(1-imidazolyl)-butyl]-2H-benzo[b]furan-3-one,

(9) 5-[cyclohexyl-(1-imidazolyl)-methyl]-1-indanone,

(10) 2-[1-(1-imidazolyl)-butyl]-4,5-dihydro-6H-benzo[b]thiophen-7-one,

(11) 5-[1-(1-imidazolyl)-1-propyl-butyl]-1-indanone,

(12) 2-[1-(1-imidazolyl)-butyl]-4,5-dihydro-6H-benzo[b]thiophen-7-one,

(13) 2-[1-(1-imidazolyl)-butyl]-4,5-dihydro-6-oxo-cyclopenta[b]-thiophene,

(14) 5-(1-imidazolylmethyl)-1-indanone,

(15) 5-[1-(1,2,4-triazolyl)-methyl]-1-indanone.

The compounds of formula I as disclosed in DE-A-3 926 365. These are especially the compounds of formula I

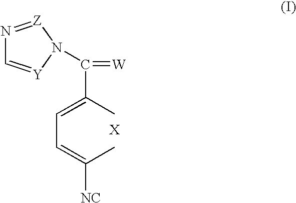

wherein W′ is a cyclopentylidene, cyclohexylidene, cycloheptylidene or 2-adamantylidene radical, X is the grouping —CH═CH—, an oxygen or a sulfur atom, and Y and Z, independently of one another, are each a methine group (CH) or a nitrogen atom, and their pharmaceutically acceptable addition salts with acids.

Individual compounds from that group that may be given special mention are:

(1) 4-[1-cyclohexylidene-1-(imidazolyl)-methyl]-benzonitrile,

(2) 4-[1-cyclopentylidene-1-(imidazolyl)-methyl]-benzonitrile,

(3) 4-[1-cycloheptylidene-1-(imidazolyl )-methyl]-benzonitrile,

(4) 4-[2-adamantylidene-1-(imidazolyl)-methyl]-benzonitrile,

(5) 4-[1-cyclohexylidene-1-(1,2,4-triazolyl)-methyl]-benzonitrile,

(6) 4-[1-cyclopentylidene-1-(1,2,4-triazolyl)-methyl]-benzonitrile,

(7) 4-[1-cycloheptylidene-1-(1,2,4-triazolyl)-methyl]-benzonitrile,

(8) 4-[2-adamantylidene-1-(1,2,4-triazolyl )-methyl]-benzonitrile,

(9) 4-[1-cyclohexylidene-1-(1,2,3-triazolyl)-methyl]-benzonitrile,

(10) 4-[1-cyclopentylidene-1-(1,2,3-triazolyl)-methyl]-benzonitrile,

(11) 5-[cyclohexylidene-1-imidazolylmethyl]-thiophene-2-carbonitrile.

The compounds of formula I as defined in DE-A-3 740 125. These are especially the compounds of formula I

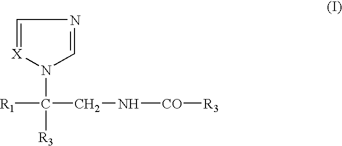

wherein X is CH or N, R₁ and R₂ are identical or different and are each phenyl or halophenyl, and R₃ is C₁-C₄ alkyl; C₁-C₄ alkyl substituted by CN, C₁-C₄ alkoxy, benzyloxy or by C₁-C₄ alkoxy-(mono-, di- or tri-)ethyleneoxy; C₁-C₄ alkoxy, phenyl; phenyl that is substituted by halogen or by cyano; a C₅-C₇ cycloalkyl group that is optionally condensed by benzene, or is thienyl, pyridyl or 2- or 3-indolyl; and acid addition salts thereof.

An individual compound from that group that may be given special mention is:

2,2-bis(4-chlorophenyl)-2-(1H-imidazol-1-yl)-1-(4-chlorobenzoyl-amino)ethane.

The compounds of formula I as defined in EP-A-293 978. These are especially the compounds of formula I

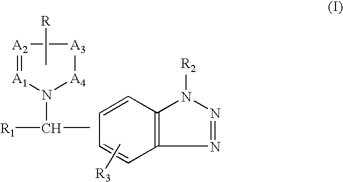

pharmaceutically acceptable salts and stereochemically isomeric forms thereof, wherein -A₁=A₂A₃=A₄- is a divalent radical selected from —CH═N—CH═CH—, —CH—N—CH═N— and —CH═N—N═CH—, R is hydrogen or C₁-C₆ alkyl; R₁ is hydrogen, C₁-C₁₀ alkyl, C₃-C₇ cycloalkyl, Ar₁, Ar₂—C₁-C₆ alkyl, C₂-C₆ alkenyl or C₂-C₆ alkynyl: R₂ is hydrogen; C₁-C₁₀ alkyl that is unsubstituted or substituted by Ar₁; C₃-C₇ cycloalkyl, hydroxy, C₁-C₆ alkoxy, Ar₁, C₂-C₆ alkenyl, C₂-C₆ alkynyl, C₃-C₇ cycloalkyl, bicyclo[2.2.1]heptan-2-yl, 2,3-dihydro-1H-indenyl, 1,2,3,4-tetrahydronaphthyl, hydroxy; C₂-C₆ alkenyloxy that is unsubstituted or substituted by Ar₂; C₂-C₆ alkynyloxy; pyrimidyloxy; di(Ar2)methoxy, (1-C₁-C₄ alkyl-4-piperidinyl)oxy, C₁-C₁₀ alkoxy; or C₁-C₁₀ alkoxy that is substituted by halogen, hydroxy, C₁-C₆ akloxy, amino, mono- or di-(C₁-C₆ alkyl)amino, trifluoromethyl, carboxy, C₁-C₆ alkoxycarbonyl, Ar.sub.1, Ar₂—O—, Ar₂-S—, C₃-C₇ cycloalkyl, 2,3-dihydro-1,4-benzodioxinyl, 1H-benzimidazolyl, C₁-C₄ alkyl-substituted 1H-benzimidazolyl, (1,1′-biphenyl)-4-yl or by 2,3-dihydro-2-oxo-1H-benzimidazolyl; and R₃ is hydrogen, nitro, amino, mono- or di-(C₁-C₆ alkyl)amino, halogen, C₁-C₆ alkyl, hydroxy or C₁-C₆ alkoxy; wherein Ar₁ is phenyl, substituted phenyl, naphthyl, pyridyl, aminopyridyl, imidazolyl, triazolyl, thienyl, halothienyl, furanyl, C₁-C₆ alkylfuranyl, halofuranyl or thiazolyl; wherein Ar₂ is phenyl, substituted phenyl or pyridyl; and wherein “substituted phenyl” is phenyl that is substituted by up to 3 substituents in each case selected independently of one another from the group consisting of halogen, hydroxy, hydroxymethyl, trifluoromethyl, C₁-C₆ alkyl, C₁-C₆ alkoxy, C₁-C₆ alkoxycarbonyl, carboxy, formyl, hydroxyiminomethyl, cyano, amino, mono- and di-(C₁-C₆ alkyl)amino and nitro.

Individual compounds from that group that may be given special mention are:

(1) 6-[(1H-imidazol-1-yl)-phenylmethyl]-1-methyl-1H-benzotriazole,

(2) 6-[(4-chlorophenyl)(1H-1,2,4-triazol-1-yl)methyl]-1-methyl-1H-benzotriazole.

The compounds of formula II as defined in EP-A-250 198, especially

(1) 2-(4-chlorophenyl)-1,1-di(1,2,4-triazol-1-ylmethyl)ethanol,

(2) 2-(4-fluorophenyl)-1,1-di(1,2,4-triazol-1-ylmethyl)ethanol,

(3) 2-(2-fluoro-4-trifluoromethylphenyl)-1,1-di(1,2,4-triazol-1-ylmethyl)ethanol,

(4) 2-(2,4-dichlorophenyl)-1,1-di(1,2,4-triazol-1-ylmethyl)ethanol,

(5) 2-(4-chlorophenyl)-1,1-di(1,2,4-triazol-1-ylmethyl)-ethanol,

(6) 2-4-fluorophenyl)-1,1-di(1,2,4-triazol-1-yl-methyl)ethanol.

The compounds of formula I as defined in EP-A-281 283, especially

(1R*2R*)-6-fluoro-2-(4-fluorophenyl)-1,2,3,4-tetrahydro-1-(1H-1,2,4-triazol-1-yl-methyl)naphthalene,

(1R *,2R * )-6-fluoro-2-(4-fluorophenyl)-1,2,3,4-tetrahydro-1-(1H-imidazolylmethyl)-naphthalene,

(1R*,2R*)- and (1R*,2S*)-2-(4-fluorophenyl)-1,2,3,4-tetrahydro-1-(1H-1,2,4-triazol-1-ylmethyl)naphthalene-6-carbonitrile,

(1R*,2R*)- and (1R*,2S*)-2-(4-fluorophenyl)-1,2,3,4-tetrahydro-1-(1H-imidazolylmethyl)naphthalene-6-carbonitrile,

(1R*,2R*)- and (1R*,2S*)-1,2,3,4-tetrahydro-1-(1H-1,2,4-triazol-1-ylmethyl)-naphthalene-2,6-dicarbonitrile,

(1R*,2R*)- and (1R*,2S*)-1,2,3,4-tetrahydro-1-(1H-imidazol-1-ylmethyl)naphthalene-2,6-dicarbonitrile,

(1R*,2S*)-2-(4-fluorophenyl)-1,2,3,4-tetrahydro-1-(5-methyl-1H-imidazolyl-methyl)naphthalene-6-carbonitrile.

The compounds of formula I as defined in EP-A-296 749, especially

2,2′-[5-(1H-1,2,4-triazol-1-ylmethyl)-1,3-phenylene]di(2-methylpropiononitrile),

2,2′-[5-(imidazol-1-ylmethyl)-1,3-phenylene]di(2 methylpropiononitrile),

(3) 2-[3-(1-hydroxy-1-methylethyl)-5-(5H-1,2,4-triazol-1-ylmethyl)phenyl]-2-methylpropiononitrile,

2,2′-[5-dideuterio(1H-1,2,4-triazol-1-yl)methyl-1,3-phenylene]di(2-trideuteriomethyl-3,3,3-trideuteriopropiononitrile),

2,2′-[5-dideuterio(1H-1,2,4-triazol-1-yl)methyl-3-phenylene]di(2methylpropiononitile).

The compounds of formula I as defined in EP-A-299 683, especially

(Z)-α-(1,2,4-triazol-1-ylmethyl)stilbene-4,4′-dicarbonitrile,

(Z)-4′-chloro-α-(1,2,4-triazol-1-ylmethyl)stilbene-4-carbonitrile,

(Z)-α-(1,2,4-triazol-1-ylmethyl)-4′-(trifluoromethyl)stilbene-4-carbonitrile,

(E)-.beta.-fluoro-α-(1,2,4-triazol-1-ylmethyl)stilbene-4,4′-dicarbonitrile,

(Z)-4′-fluoro-α-(imidazol-1-ylmethyl)stilbene-4-carbonitrile,

(Z)-2′,4′-dichloro-α-(imidazol-1-ylmethyl)stilbene-4-carbonitrile,

(Z)-4′-chloro-α-(imidazol-1-ylmethyl)stilbene-4-carbonitrile,

(Z)-α-(imidazol-1-ylmethyl)stilbene4,4′dicarbonitrile,

(Z)-α-(5-methylimidazol-1-ylmethyl)stilbene-4,4′-dicarbonitrile,

(Z)-2-[2-(4-cyanophenyl)-3-(1,2,4-triazol-1-yl)propenyl]pyridine-5-carbonitrile.

The compounds of formula I as defined in EP-A-299 684, especially

(1) 2-(4-chlorobenzyl)-2-fluoro-1,3-di(,2,4-triazol-1-yl)propane,

(2) 2-fluoro-2-(2-fluoro-4-chlorobenzyl)-1,3-di(1,2,4-triazol-1-yl)propane,

(3) 2-fluoro-2-(2-fluoro-4-trifluoromethylbenzyl)-1,3-di(1,2,4-triazol-1-yl)propane,

(4) 3-(4-chlorophenyl)- -(1,2,4-triazol-1-yl)-2-(1,2,4-triazol-1-ylmethyl)butan-2-ol,

(5) 2-(4-chloro-α-fluorobenzyl)-1,3-di(1,2,4-triazol-1-yl)propan-2-ol,

(6) 2-(4-chlorobenzyl)-1,3-bis(1,2,4-triazol-1-yl)propane,

(7) 4-[2-(4-chlorophenyl)-1,3-di(1,2,4-triazol-1-ylmethyl)ethoxymethyl]-benzonitrile,

(8) 1-(4-fluorobenzyl)-2-(2fluoro-4-trifluoromethylphenyl)-1,3-di(1,2,4-triazol-1-yl)-propan-2-ol,

(9) 2-(4-chlorophenyl)-1-(4-fluorophenoxy)-1,3-di(1,2,4-triazol-1-yl)propan-2-ol,

(10) 1-(4-cyanobenzyl )-2-(2,4-difluorophenyl)-1,3di(1,2,4-triazol-1-yl)propan-2-ol,

(11) 2-(4-chlorophenyl)-1-phenyl-1,3-di(1,2,4-triazol-1-yl)propan-2-ol.

The compounds as defined in claim 1 of EP-A-316 097, especially

1,1-dimethyl-8-(1H-1,2,4-triazol-1-ylmethyl)-2(1H)-naphtho[2,1-b]furanone,

1,2-dihydro1,1-dimethyl-2-oxo-8-(1H-1,2,4-triazol-l -ylmethyl)naphtho[2,1-b]-furan-7-carbonitrile,

1,2-dihydro-1,1-dimethyl-2-oxo-8-(1H-1,2,4-triazol-1-ylmethyl)naphtho[2,1-b]-furan-7-carboxamide,

1,2-dihydro-1,1-dimethyl-2-oxo-8-[di(1H-1,2,4-triazol-1-yl)methyl]naphtho[2,1-b]-furan-7-carbonitrile.

The compounds of formula I as defined in EP-A-354 689, especially

(1) 4-[2-(4-cyanophenyl)-3-(1,2,4-triazol-1-yl)propyl]benzonitrile,

(2) 4-[1-(4-chlorobenzyl)-2-(1,2,4-triazol-1-yl)ethyl]benzonitrile,

(3) 4-[2-(1,2,4-triazol-1-yl)-1-(4-trifluoromethyl]benzyl)ethyl]benzonitrile,

(4) 4-[2-(1,2,4-triazol-1-yl)-1-(4-[trifluoromethoxy]benzyl)ethyl]benzonitrile.

The compounds of formula (1) as defined in EP-A-354 683, especially

(1) 6-[2-(4-cyanophenyl)-3-(1,2,4-triazol-1-yl)-propyl]nicotinonitrile,

(2) 4-[1-(1,2,4-triazol-1-yl-methyl)-2-(5-[trifluoromethyl]pyrid-2-yl)ethyl]benzonitrile.

Examples of steroidal aromatase inhibitors that may be mentioned are:

The compounds of formula I as defined in EP-A-181 287. These are especially the compounds of formula I

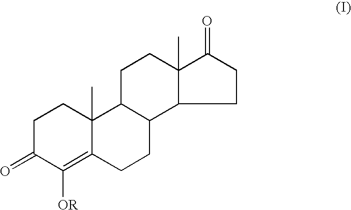

wherein R is hydrogen, acetyl, heptanoyl or benzoyl. An individual compound from that group that may be given special mention is:

(1) 4-hydroxy-4-androstene-3,17-dione.

(ab) The compounds as defined in the claims of U.S. Pat. No. 4,322,416, especially 10-(2-propynyl)-oestr-4-ene-3,17-dione.

(ac) The compounds as defined in the claims of DE-A-3 622 841, especially 6-methyleneandrosta-1,4-diene-3,17-dione.

(ad) The compounds as defined in the claims of GB-A-2 17 1100, especially 4-amino-androsta-1,4,6-triene-3,17-dione.

Also: (ae) androsta-1,4,6-triene-3,17-dione.

The content of the patent applications mentioned under (a) to (z) and (aa) to (ad), especially the subgroups of compounds disclosed therein and the individual compounds disclosed therein as examples, have been incorporated by reference into the disclosure of the present application.

The general terms used hereinbefore and hereinafter to define the compounds have the following meanings:

Organic radicals designated by the term “lower” contain up to and including 7, preferably up to and including 4, carbon atoms.

Acyl is especially lower alkanoyl.

Aryl is, for example, phenyl or 1- or 2-naphthyl, each of which is unsubstituted or substituted by lower alkyl, hydroxy, lower alkoxy, lower alkanoyloxy, amino, lower alkylamino, di-lower alkylamino, lower alkanoylamino or by halogen.

Pharmaceutically acceptable salts of the above-mentioned compounds are, for example, pharmaceutically acceptable acid addition salts or pharmaceutically acceptable metal or ammonium salts.

Pharmaceutically acceptable acid addition salts are especially those with suitable inorganic or organic acids, for example strong mineral acids, such as hydrochloric acid, sulfuric acid or phosphoric acid, or organic acids, especially aliphatic or aromatic carboxylic or sulfonic acids, for example formic, acetic, propionic, succinic, glycolic, lactic, hydroxysuccinic, tartaric, citric, maleic, fumaric, hydroxymaleic, pyruvic, phenylacetic, benzoic, 4-aminobenzoic, antbranilic, 4-hydroxybenzoic, salicylic, 4-aminosalicylic, pamoic, gluconic, nicotinic, methanesulfonic, ethanesulfonic, halobenzenesulfonic, p-toluenesulfonic, naphthalenesulfonic, sulfanilic or cyclohexylsulfamic acid; or with other acidic organic substances, for example ascorbic acid. Pharmaceutically acceptable salts may also be formed, for example, with amino acids, such as arginine or lysine.

Compounds containing acid groups, for example a free carboxy or sulfo group, can also form pharmaceutically acceptable metal or ammonium salts, such as alkali metal or alkaline earth metal salts, for example sodium, potassium, magnesium or calcium salts, also ammonium salts derived from ammonia or suitable organic amines. Them come into consideration especially aliphatic, cycloaliphatic, cycloaliphatic-aliphatic or araliphatic primary, secondary or tertiary mono-, di- or poly-amines, such as lower alkylamines, for example di- or tri-ethylamine, hydroxy-lower alkylamines, for example 2-hydroxyethylamine, bis(2-hydroxyethyl)amine or tris(2-hydroxyethyl)amine, basic aliphatic esters or carboxylic acids, for example 4-aminobenzoic acid 2-diethylaminoethyl ester, lower alkyleneamines, for example 1-ethylpiperidine, cycloalkylamines, for example dicyclohexylamine, benzylamines, for example N,N′-dibenzylethylenediamine; also heterocyclic bases, for example of the pyridine type, for example pyridine, collidine or quinolne. If several acidic or basic groups are present, mono- or poly-salts can be formed. Compounds according to the invention having an acidic and a basic group may also be in the form of internal salts, i.e. in the form of zwitterions and another part of the molecule in the form of a normal salt.

In the case of the above-mentioned individual compounds the pharmaceutically acceptable salts are included in each case insofar as the individual compound is capable of salt formation.

The compounds listed, including the individual compounds mentioned, both in free form and in salt form, may also be in the form of hydrates, or their crystals may include, for example, the solvent used for crystallisation. The present invention relates also to all those forms.

Many of the above-mentioned compounds, including the individual compounds mentioned, contain at least one asymmetric carbon atom. They can therefore occur in the form of R— or S-enantiomers and as enantiomeric mixtures thereof, for example in the form of a racemate. The present invention relates to the use of all those forms and to the use of all further isomers, and of mixtures of at least 2 isomers, for example mixtures of diastereoisomers or enantiomers which can occur when there are one or more further asymmetric centres in the molecule. Also included are, for example, all geometric isomers, for example cis- and trans-isomers, that can occur when the compounds contain one or more double bonds.

A Physiologic Replacement Dose of Estrogen

A physiologic replacement dose of estrogen is a dose required to bring serum estradiol levels to approximately the level found in a healthy reproductive age male. If another estrogen is used, the equivalent replacement dose will be known by the skilled practitioner. Serum estradiol levels should preferably be brought to the range at or about 10-75 pg/ml, more preferably at or about 15-50 pg/ml and most preferably at or about 25 pg/ml.

Preferred doses are as follows:

| Estrogen | Dose Ranges |
|---|---|
| Transdermal estradiol | at or about 10-50 μg twice weekly |
| | prefereably at or about 10-25 μg twice weekly |
| | more preferably at or about 12.5 to 25 μg twice |
| | weekly |
| | most preferably at or about 25 μg twice weekly |
| Oral estradiol | at or about 100-1,000 μg daily |
| | preferably at or about 100-500 μg daily |
| | more preferably at or about 250-500 μg daily |
| | most preferably at or about 250 μg daily |

Any substance that exhibits appropriate estrogenic activity may be used in the present invention. As indicated the preferred estrogen is 17β-estradiol. Other suitable estrogens include, but are not limited to, estradiol valerate, other estrogens, 17α-ethinylestradiol, esters and ethers of 17α-ethinylestradiol such as, for example, 17α-ethinylestradiol 3-dimethylamino propionate, 17α-ethinylestradiol 3-cyclopentyl ether (quienestrol) and 17α-ethinylestradiol 3-methyl ether (mestranol). Natural estrogens such as estrone, estrone sulfate, estrone sulfate piperazine salt, estradiol and estriol, and their esters, conjugated equine estrogens and any of its components demonstrating estrogenic or antioxidant activity, as well as the synthetic estrogens, may also be employed. The selection of the estrogen and the dose level will generally follow from the literature, which is well known to the person skilled in the art.

Dose of Aromatase Inhibitor

The dose of the aromatase inhibitor will be tailored to the particular patient (as well the dose of estrogen). The patient can be started on a regimen (for example the bio-equivalent of at or about 0.25 mg to 10 mg Anastrozle daily and the bio-equivalent of at or about 0.125 to 1.0 mg or about 0.125 to 0.5 mg per day of estradiol), and the doses adjusted until the patient reports an improvement in libido and/or in mood.

The dose of aromatase inhibitor will preferably be such that it results in an increase of androgen serum levels over the basal level for the patient in question. In a male, it is preferred that androgen levels reach at least at or about 350 to 1000 ng/dL, more preferably at or about 400 to 700 ng/dL.

Letrozole and anastrazole are preferred aromatase inhibitors. Other suitable aromatase inhibitors include but are not limited to exemestane, vorozole, fadrozole, pentrozole, formestane, atamestane and testolactone. If anastrozole (Arimidex) is used, a preferred dose is selected from at or about 0.25 to at or about 10 mg. If another aromatase inhibitor is used, the preferred dose may be defined as a bio-equivalent dose to the dose range for anastrozole. For example, the preferred dose for letrozole is also between at or about 0.25 to at or about 10 mg per day. The preferred dose for exemestane is between at or about 5 mg to at or about 50 mg per day. The preferred dose for testolactone is between at or about 100 mg to at or about 400 mg daily.

Pharmaceutical Formulations

The pharmaceutical compositions that can be prepared according to the invention are compositions for enteral, such as peroral or rectal administration, also for transdermal or sublingual administration, and for parenteral, for example intravenous, subcutaneous and intramuscular, administration. Suitable unit dose forms, especially for peroral and/or sublingual administration, for example dragees, tablets or capsules, comprise preferably from approximately 0.01 mg to approximately 20 mg, especially from approximately 0.1 mg to approximately 10 mg, of the combination of the above-mentioned compounds or of a pharmaceutically acceptable salt thereof, together with pharmaceutically acceptable carriers. The preferred form of administration is oral. The proportion of active ingredient in such pharmaceutical compositions is generally from approximately 0.001% to approximately 60%, preferably from approximately 0.1% to approximately 20%.

Suitable excipients for pharmaceutical compositions for oral administration are especially fillers, such as sugars, for example lactose, saccharose, mannitol or sorbitol, cellulose preparations and/or calcium phosphates, for example tricalcium phosphate or calcium hydrogen phosphate, and binders, such as starches, for example corn, wheat, rice or potato starch, gelatin, tragacanth, methylcellulose and/or hydroxypropylcellulose, disintegrators, such as the above-mentioned starches, also carboxymethyl starch, cross-linked polyvinylpyrrolidone, agar, alginic acid or a salt thereof, such as sodium alginate, and/or cellulose, for example in the form of crystals, especially in the form of microcrystals, and/or flow regulators and lubricants, for example silicic acid, talc, stearic acid or salts thereof, such as magnesium or calcium stearate, cellulose and/or polyethylene glycol.

Dragee cores can be provided with suitable, optionally enteric, coatings, there being used inter alia concentrated sugar solutions which may comprise gum arabic, talc, polyvinylpyrrolidone, polyethylene glycol and/or titanium dioxide, or coating solutions in suitable solvents or solvent mixtures, or, for the preparation of enteric coatings, solutions of suitable cellulose preparations, such as acetylcellulose phthalate or hydroxypropylmethylcellulose phthalate.

Other orally administrable pharmaceutical compositions are dry-filled capsules consisting of gelatin, and also soft sealed capsules consisting of gelatin and a plasticiser, such as glycerol or sorbitol. The dry-filled capsules may contain the active ingredient in the form of granules, for example in admixture with fillers, such as lactose, binders, such as starches, and/or glidants, such as talc or magnesium stearate, and, if desired, stabilisers. In soft capsules, the active ingredient is preferably dissolved or suspended in suitable oily excipients, such as fatty oils, paraffin oil or liquid polyethylene glycols, to which stabilisers and/or anti-bacterial agents may also be added.

There may also be used capsules that are easily bitten through, in order to achieve by means of the sublingual ingestion of the active ingredient that takes place as rapid an action as possible.

Suitable rectally administrable pharmaceutical compositions are, for example, suppositories that consist of a combination of the active ingredient with a suppository base. Suitable suppository bases are, for example, natural or synthetic triglycerides, paraffin hydrocarbons, polyethylene glycols or higher alkanols. There may also be used gelatin rectal capsules, which contain a combination of the active ingredient with a base material. Suitable base materials are, for example, liquid triglycerides, polyethylene glycols or paraffin hydrocarbons.

Suitable formulations for transdermal administration comprise the active ingredient together with a carrier. Advantageous carriers include absorbable pharmacologically acceptable solvents that serve to facilitate the passage through the skin of the host. Transdermal systems are usually in the form of a bandage that comprises a support, a supply container containing the active ingredient, if necessary together with carriers, optionally a separating device that releases the active ingredient onto the skin of the host at a controlled and established rate over a relatively long period of time, and means for securing the system to the skin.

Suitable for parenteral administration are especially aqueous solutions of an active ingredient in water-soluble form, for example in the form of a water-soluble salt, and also suspensions of active ingredient, such as corresponding oily injection suspensions, there being used suitable lipophilic solvents or vehicles, such as fatty oils, for example sesame oil, or synthetic fatty acid esters, for example ethyl oleate, or triglycerides, or aqueous injection suspensions that comprise viscosity-increasing substances, for example sodium carboxymethylcellulose, sorbitol and/or dextran, and, optionally, stabilisers.

Dyes or pigments may be added to the pharmaceutical compositions, especially to the tablets or dragee coatings, for example for identification purposes or to indicate different doses of active ingredient.

The pharmaceutical compositions of the present invention can be prepared in a manner known per se, for example by means of conventional mixing, granulating, confectioning, dissolving or lyophilising processes. For example, pharmaceutical compositions for oral administration can be obtained by combining the active ingredient with solid carriers, optionally granulating a resulting mixture, and processing the mixture or granules, if desired or necessary after the addition of suitable excipients, to form tablets or dragee cores.

The benefits of this invention compared to present treatments for androgen deficiency include, 1) The stable increase of endogenous testosterone will prevent the need for exogenous testosterone administration which is associated with supraphysiologic peaks of testosterone, and resulting fluctuations in androgen levels. 2) Avoidance of painful testosterone injections, skin irritation from transdermal testosterone patches, or potential liver toxicity from oral testosterone administration. 3) Low dose estrogen administration may improve the lipid profile, specifically increasing HDL-C (High-Density Lipoprotein Cholesterol), in contrast to testosterone injections, which are associated with supraphysiologic peaks of testosterone and decreased HDL-C. 4) Low dose estrogen may also improve vascular flow by a direct action on the blood vessel wall thereby reducing the risk of cardiovascular disease.

An example of a suitable pharmaceutical preparation for oral administration to a male in need of hormone replacement therapy may comprise micronized estradiol between about 0.1 mg to about 1.0 mg combined with anastrazole between about 0.25 mg to about 10.0 mg.

An alternative pharmaceutical preparation for oral administration to a male in need of hormone replacement therapy may comprise micronized estradiol between about 0.125 mg to about 0.5 mg combined with anastrazole between about 0.25 mg to about 10.0 mg, preferably between at or about 0.25mg to at or about 2.0 mg anastrazole.

An alternative pharmaceutical preparation for oral administration to a male in need of hormone replacement therapy may comprise micronized estradiol between about 0.25 mg to about 0.5 mg combined with anastrazole between about 0.25 mg to 2.0 mg, preferably at or about 0.25mg to at or about 1.0 mg anastrazole.

Another alternative pharmaceutical preparation for oral administration to a male in need of hormone replacement therapy may comprise micronized estradiol at or about 0.25 mg combined with about 0.5 mg anastrazole.

Improvement in libido is typically evaluated by interviewing the patient, and asking the patient keep a written record over a given period, keeping note of such things as number of acts of sexual intercourse, masturbation, sexual fantasies, and erections.

Examples of methods of evaluating libido and mood in males are found in the following references: Bagatell et al.; J. Clin. Endocrinol. & Metabol. 1994 78:711-716; Davidson et al.; Arch. Sexual Behaviour 1983 12:263-274; Gooren; Arch. Sexual Behaviour 1985 14:539-548; Carani et al.; Clin. Endocrinol. 1999 51:517-524.

While the invention has been described with particular reference to certain embodiments thereof, it will be understood that changes and modifications may be made by those of ordinary skill in the art within the scope and spirit of the following claims.

In the claims, the word “comprising” means “including the following elements (in the body), but not excluding others”; the phrase “consisting of” means “excluding more than traces of other than the recited ingredients”; and the phrase “consisting essentially of” means “excluding unspecified ingredients which materially affect the basic characteristics of the composition”.

I claim:

1. A pharmaceutical preparation for administration to a male in need of hormone replacement therapy, comprising a plurality of doses for administration, each dose comprising at least one aromatase inhibitor and an estrogen.

2. A pharmaceutical preparation according to claim 1, wherein the estrogen is present in a physiologic replacement dose.

3. A pharmaceutical preparation according to claim 1, wherein the aromatase inhibitor is present in an amount that is bio-equivalent to at or about 0.25 to at or about 10 mg of anastrazole per day.

4. A pharmaceutical preparation for administration to a male in need of hormone replacement therapy, comprising a plurality of doses for administration, each dose comprising at least one aromatase inhibitor bio-equivalent to between at or about 0.25 to at or about 10.0 mg per day of anastrazole and a dose of estrogen bio-equivalent to between at or about 0.125 to at or about 1.0 mg per day of estradiol.

5. A pharmaceutical preparation as claimed in claim 4, comprising a plurality of doses for administration, each dose comprising at least one aromatase inhibitor bio-equivalent to between at or about 0.25 to at or about 10.0 mg per day of anastrazole and a dose of estrogen bio-equivalent to between at or about 0.125 to at or about 0.5 mg per day of estradiol.

6. A pharmaceutical preparation as claimed in claim 5 comprising a plurality of doses for administration, each dose comprising at least one aromatase inhibitor bio-equivalent to between at or about 0.50 to at or about 1.0 mg per day of anastrazole and a dose of estrogen bio-equivalent to between at or about 0.125 to at or about 0.5 mg per day of estradiol.

7. A pharmaceutical preparation as claimed in claim 6 wherein each dose comprises at least one aromatase inhibitor bio-equivalent to at or about 0.5 mg per day of anastrazole and a dose of estrogen bio-equivalent to at or about 0.25 mg per day of estradiol.

8. A pharmaceutical preparation as claimed in claim 4, wherein the estrogen is selected from estradiol valerate, 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

9. A pharmaceutical preparation as claimed in claim 4, wherein the aromatase inhibitor is selected from aromatase inhibitors having a half-life of at or about 8 hours to at or about 4 days.

10. A pharmaceutical preparation as claimed in claim 4, wherein the aromatase inhibitor is selected from aromatase inhibitors having a half-life of at or about 2 days.

11. A pharmaceutical preparation as claimed in claim 4, wherein the aromatase inhibitor is selected from at least one of non-steroidal and reversible aromatase inhibitors.

12. A pharmaceutical preparation as claimed in claim 4, wherein the aromatase inhibitor is a non-reversible aromatase inhibitor.

13. A pharmaceutical preparation as claimed in claim 4, wherein the pharmaceutical preparation is for oral administration.

14. A pharmaceutical preparation for administration to a male in need of treatment for a mood disorder, comprising a plurality of doses for administration, each dose comprising at least one aromatase inhibitor and an estrogen.

15. A pharmaceutical preparation as claimed in claim 13, wherein the estrogen is present in a physiologic replacement dose.

16. A pharmaceutical preparation as claimed in claim 13, wherein the aromatase inhibitor is present in an amount that is bio-equivalent to at or about 0.25 to at or about 10 mg of Anastrozole per day.

17. A pharmaceutical preparation as claimed in claim 13, wherein the mood disorder is loss of libido.

18. A pharmaceutical preparation as claimed in claim 13, wherein the mood disorder is depression.

19. A pharmaceutical preparation for administration to a male in need of treatment for a mood disorder, comprising a plurality of doses for administration, each dose comprising at least one aromatase inhibitor bio-equivalent to between at or about 0.25 to at or about 10.0 mg per day of anastrazole and a dose of estrogen bio-equivalent to between at or about 0.125 to at or about 1.0 mg per day of estradiol.

20. A pharmaceutical preparation as claimed in claim 19, comprising a plurality of doses for administration, each dose comprising at least one aromatase inhibitor bio-equivalent to between at or about 0.25 to at or about 10.0 mg per day of anastrazole and a dose of estrogen bio-equivalent to between at or about 0.125 to at or about 0.5 mg per day of estradiol.

21. A pharmaceutical preparation as claimed in claim 20 comprising a plurality of doses for administration, each dose comprising at least one aromatase inhibitor bio-equivalent to between at or about 0.50 to at or about 1.0 mg per day of anastrazole and a dose of estrogen bio-equivalent to between at or about 0.125 to at or about 0.5 mg per day of estradiol.

22. A pharmaceutical preparation as claimed in claim 19 wherein each dose comprises at least one aromatase inhibitor bio-equivalent to at or about 0.5 mg per day of anastrazole and a dose of estrogen bio-equivalent to at or about 0.25 mg per day of estradiol.

23. A pharmaceutical preparation as claimed in claim 19, wherein the estrogen is selected from estradiol valerate, 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

24. A pharmaceutical preparation as claimed in claim 19, wherein the aromatase inhibitor is selected from aromatase inhibitors having a half-life of at or about 8 hours to at or about 4 days.

25. A pharmaceutical preparation as claimed in claim 19, wherein the aromatase inhibitor is selected from aromatase inhibitors having a half-life of at or about 2 days.

26. A pharmaceutical preparation as claimed in claim 19, wherein the aromatase inhibitor is selected from at least one of non-steroidal and reversible aromatase inhibitors.

27. A pharmaceutical preparation as claimed in claim 19, wherein the aromatase inhibitor is a non-reversible aromatase inhibitor.

28. A pharmaceutical preparation as claimed in claim 19, wherein the pharmaceutical preparation is for oral administration.

29. A package containing a pharmaceutical preparation comprising a plurality of doses for administration, each dose comprising at least one aromatase inhibitor and an estrogen, and instructions for use of the preparation in the treatment of a male in need of hormone replacement therapy.

30. A package containing a pharmaceutical preparation comprising a plurality of doses for administration, each dose comprising at least one aromatase inhibitor bio-equivalent to at or between about 0.25 to at or about 10.0 mg per day of anastrazole and a dose of estrogen bio-equivalent to between at or about 0.125 to at or about 1.0 mg estradiol per day, and instructions for use of the preparation in the treatment of a male in need of hormone replacement therapy.

31. A package containing a pharmaceutical preparation as claimed in claim 30 wherein each dose comprises at least one aromatase inhibitor bio-equivalent to at or between about 0.25 to at or about 10.0 mg per day of anastrazole and a dose of estrogen bio-equivalent to between at or about 0.125 to at or about 0.5 mg estradiol per day, and instructions for use of the preparation in the treatment of a male in need of hormone replacement therapy.

32. A package containing a pharmaceutical preparation as claimed in claim 31 wherein each dose comprises at least one aromatase inhibitor bio-equivalent to at or between at or about 0.50 to at or about 1.0 mg per day of anastrazole and a dose of estrogen bio-equivalent to between at or about 0.125 to at or about 0.5 mg per day of estradiol.

33. A package containing a pharmaceutical preparation as claimed in claim 32 wherein each dose comprises at least one aromatase inhibitor bio-equivalent to at or about 0.5 mg per day of anastrazole and a dose of estrogen bio-equivalent to at or about 0.25 mg per day of estradiol.

34. A package containing a pharmaceutical preparation as claimed in claim 30, wherein the estrogen is selected from 17α-ethinylestradiol, esters and ethers of 17a-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

35. A package containing a pharmaceutical preparation as claimed in claim 30, wherein the aromatase inhibitor is selected from aromatase inhibitors having a half-life of about 8 hours to about 4 days.

36. A package containing a pharmaceutical preparation as claimed in claim 30, wherein the aromatase inhibitor is selected from aromatase inhibitors having a half-life of about 2 days.

37. A package containing a pharmaceutical preparation as claimed in claim 30, wherein the aromatase inhibitor is selected from at least one of non-steroidal and reversible aromatase inhibitors.

38. A package containing a pharmaceutical preparation as claimed in claim 30, wherein the aromatase inhibitor is non-reversible.

39. A package containing a pharmaceutical preparation as claimed in claim 30, wherein the pharmaceutical preparation is administered orally.

40. A package containing a pharmaceutical preparation comprising a plurality of doses for administration, each dose comprising at least one aromatase inhibitor and an estrogen, and instructions for use of the preparation in the treatment of a male in need of treatment for a mood disorder.

41. A package containing a pharmaceutical preparation comprising a plurality of doses for administration, each dose comprising at least one aromatase inhibitor bio-equivalent to at or between about 0.25 to at or about 10.0 mg per day of anastrazole and a dose of estrogen bio-equivalent to between at or about 0.125 to at or about 1.0 mg estradiol per day, and instructions for use of the preparation in the treatment of a male in need of treatment for a mood disorder.

42. A package containing a pharmaceutical preparation as claimed in claim 41 wherein each dose comprises at least one aromatase inhibitor bio-equivalent to at or between about 0.25 to at or about 10.0 mg per day of anastrazole and a dose of estrogen bio-equivalent to between at or about 0.125 to at or about 0.5 mg estradiol per day, and instructions for use of the preparation in the treatment of a male in need of treatment for a mood disorder.

43. A package containing a pharmaceutical preparation as claimed in claim 42 wherein each dose comprises at least one aromatase inhibitor bio-equivalent to between at or about 0.50 to at or about 1.0 mg per day of anastrazole and a dose of estrogen bio-equivalent to between at or about 0.125 to at or about 0.5 mg estradiol per day of estradiol.

44. A package containing a pharmaceutical preparation as claimed in claim 43 wherein each dose comprises at least one aromatase inhibitor bio-equivalent to at or about 0.5 mg per day of anastrazole and a dose of estrogen bio-equivalent to at or about 0.25 mg per day of estradiol.

45. A package containing a pharmaceutical preparation as claimed in claim 41, wherein the estrogen is selected from 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

46. A package containing a pharmaceutical preparation as claimed in claim 41, wherein the aromatase inhibitor is selected from aromatase inhibitors having a half-life of about 8 hours to about 4 days.

47. A package containing a pharmaceutical preparation as claimed in claim 41, wherein the aromatase inhibitor is selected from aromatase inhibitors having a half-life of about 2 days.

48. A package containing a pharmaceutical preparation as claimed in claim 41, wherein the aromatase inhibitor is selected from at least one of non-steroidal and reversible aromatase inhibitors.

49. A package containing a pharmaceutical preparation as claimed in claim 41, wherein the aromatase inhibitor is non-reversible.

50. A package containing a pharmaceutical preparation as claimed in claim 41, wherein the pharmaceutical preparation is administered orally.

51. A method of treating a male in need of hormone replacement therapy-comprising administering to said male at least one aromatase inhibitor and an estrogen.

52. A method of treating a male in need of hormone replacement therapy comprising administering to said male at least one aromatase inhibitor bio-equivalent to between about 0.25 to about 10.0 mg per day of anastrazole and a dose of estrogen bio-equivalent to between at or about 0.125 to at or about 1.0 mg per day of estradiol.

53. A method as claimed in claim 52 comprising administering to said male at least one aromatase inhibitor bio-equivalent to between about 0.25 to about 10.0 mg per day of anastrazole and a dose of estrogen bio-equivalent to between at or about 0.125 to at or about 0.5 mg per day of estradiol.

54. A method as claimed in claim 53 comprising administering to said male a pharmaceutical regimen comprising a plurality of doses, each dose comprising at least one aromatase inhibitor bio-equivalent to between at or about 0.50 to at or about 1.0 mg per day of anastrazole and a dose of estrogen bio-equivalent to between at or about 0.125 to at or about 0.5 mg per day of estradiol.

55. A method as claimed in claim 54 comprising administering to said male a pharmaceutical regimen comprising a plurality of doses, each dose comprising at least one aromatase inhibitor bio-equivalent to at or about 0.5 mg per day of anastrazole and a dose of estrogen bio-equivalent to at or about 0.25 mg per day of estradiol.

56. A method as claimed in claim 52, wherein the physiologic replacement dose of estrogen is selected from 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

57. A method as claimed in claim 52, wherein the aromatase inhibitor is selected from aromatase inhibitors having a half-life of about 8 hours to about 4 days.

58. A method as claimed in claim 52, wherein the aromatase inhibitor is selected from aromatase inhibitors having a half-life of about 2 days.

59. A method as claimed in claim 52, wherein the aromatase inhibitor is selected from at least one of non-steroidal and reversible aromatase inhibitors.

60. A method as claimed in claim 52, wherein the aromatase inhibitor is non-reversible.

61. A method as claimed in claim 52, wherein the pharmaceutical regimen is administered orally.

62. A method of treating a male in need of treatment for a mood disorder comprising administering to said male at least one aromatase inhibitor and an estrogen.

63. A method of treating a male in need of treatment for a mood disorder comprising administering to said male at least one aromatase inhibitor bio-equivalent to between about 0.25 to about 10.0 mg per day of anastrazole and a dose of estrogen bio-equivalent to between at or about 0.125 to at or about 1.0 mg per day of estradiol.

64. A method as claimed in claim 63 comprising administering to said male at least one aromatase inhibitor bio-equivalent to between about 0.25 to about 10.0 mg per day of anastrazole and a dose of estrogen bio-equivalent to between at or about 0.125 to at or about 0.5 mg per day of estradiol.

65. A method as claimed in claim 64 comprising administering to said male a pharmaceutical regimen comprising a plurality of doses, each dose at least one aromatase inhibitor bio-equivalent to between at or about 0.50 to at or about 1.0 mg per day of anastrazole and a dose of estrogen bio-equivalent to between at or about 0.125 to at or about 0.5 mg per day of estradiol.

66. A method as claimed in claim 65 comprising administering to said male a pharmaceutical regimen comprising a plurality of doses, each dose at least one aromatase inhibitor bio-equivalent to at or about 0.5 mg per day of anastrazole and a dose of estrogen bio-equivalent to at or about 0.25 mg per day of estradiol.

67. A method as claimed in claim 63, wherein the physiologic replacement dose of estrogen is selected from 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

68. A method as claimed in claim 63, wherein the aromatase inhibitor is selected from aromatase inhibitors having a half-life of about 8 hours to about 4 days.

69. A method as claimed in claim 63, wherein the aromatase inhibitor is selected from aromatase inhibitors having a half-life of about 2 days.

70. A method as claimed in claim 63, wherein the aromatase inhibitor is selected from at least one of non-steroidal and reversible aromatase inhibitors.

71. A method as claimed in claim 63, wherein the aromatase inhibitor is non-reversible.

72. A method as claimed in claim 63, wherein the pharmaceutical regimen is administered orally.

73. A method as claimed in claim 63, wherein the mood disorder is loss of libido.

74. A method as claimed in claim 63, wherein the mood disorder is depression.

75. The use of at least one aromatase inhibitor and an estrogen in the preparation of a medicament is for administration to a male in need of hormone replacement therapy, the medicament comprising a plurality of doses each dose comprising at least one aromatase inhibitor and an estrogen.

76. The use of at least one aromatase inhibitor and an estrogen in the preparation of a medicament, characterized in that the medicament is for administration to a male in need of hormone replacement therapy, the medicament comprising a plurality of doses, each dose comprising at least one aromatase inhibitor bio-equivalent to between at or about 0.25 to at or about 10.0 mg per day of anastrazole and a dose of estrogen bio-equivalent to between at or about 0.125 to at or about 1.0 mg per day of estradiol.

77. The use as claimed in claim 76 wherein each dose comprises at least one aromatase inhibitor bio-equivalent to between at or about 0.25 to at or about 10.0 mg per day of anastrazole and a dose of estrogen bio-equivalent to between at or about 0.125 to at or about 0.5 mg per day of estradiol.

78. The use as claimed in claim 77 wherein each dose comprises at least one aromatase inhibitor bio-equivalent to between about 0.50 to at or about 1.0 mg per day of anastrazole and a dose of estrogen bio-equivalent to between at or about 0.125 to at or about 0.5 mg per day of estradiol.

79. The use as claimed in claim 78 wherein each dose comprises at least one aromatase inhibitor bio-equivalent to at or about 0.5 mg per day of anastrazole and a dose of estrogen bio-equivalent to at or about about 0.25 mg per day of estradiol.

80. The use as claimed in claim 76, wherein the estrogen is selected from 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

81. The use as claimed in claim 76, wherein the aromatase inhibitor is selected from aromatase inhibitors having a half-life of about 8 hours to about 4 days.

82. The use as claimed in claim 76, wherein the aromatase inhibitor is selected from aromatase inhibitors having a half-life of about 2 days.

83. The use as claimed in claim 76, wherein the aromatase inhibitor is selected from at least one of non-steroidal and reversible aromatase inhibitors.

84. The use as claimed in claim 76, wherein the aromatase inhibitor is non-reversible.

85. The use as claimed in claim 76, wherein the medicament is for oral administration.

86. The use of at least one aromatase inhibitor and an estrogen in the preparation of a medicament is for administration to a male in need of treatment for a mood disorder, the medicament comprising a plurality of doses each dose comprising at least one aromatase inhibitor and an estrogen.

87. The use of at least one aromatase inhibitor and an estrogen in the preparation of a medicament, characterized in that the medicament is for administration to a male in need of hormone replacement therapy, the medicament comprising a plurality of doses, each dose comprising at least one aromatase inhibitor bio-equivalent to between at or about 0.25 to at or about 10.0 mg per day of anastrazole and a dose of estrogen bio-equivalent to between at or about 0.125 to at or about 1.0 mg per day of estradiol.

88. The use as claimed in claim 87 wherein each dose comprises at least one aromatase inhibitor bio-equivalent to between at or about 0.25 to at or about 10.0 mg per day of anastrazole and a dose of estrogen bio-equivalent to between at or about 0.125 to at or about 0.5 mg per day of estradiol.

89. The use as claimed in claim 88 wherein each dose comprises at least one aromatase inhibitor bio-equivalent to between about 0.50 to at or about 1.0 mg per day of anastrazole and a dose of estrogen bio-equivalent to between at or about 0.125 to at or about 0.5 mg per day of estradiol.

90. The use as claimed in claim 89 wherein each dose comprises at least one aromatase inhibitor bio-equivalent to at or about 0.5 mg per day of anastrazole and a dose of estrogen bio-equivalent to at or about 0.25 mg per day of estradiol.

91. The use as claimed in claim 87, wherein the estrogen is selected from 17α-ethinylestradiol, esters and ethers of 17α-ethinylestradiol, 17α-ethinylestradiol 3-dimethylamino propionate, 17α-ethinylestradiol 3-cyclopentyl ether (quienestrol) and 17α-ethinylestradiol 3-methyl ether (mestranol); natural estrogens, estrone, estrone sulfate, estrone sulfate piperazine salt, estradiol and estriol, and their esters, conjugated equine estrogen and any components thereof with estrogenic or antioxidant activity, as well as the synthetic estrogens.

92. The use as claimed in claim 87, wherein the aromatase inhibitor is selected from aromatase inhibitors having a half-life of about 8 hours to about 4 days.

93. The use as claimed in claim 87, wherein the aromatase inhibitor is selected from aromatase inhibitors having a half-life of about 2 days.

94. The use as claimed in claim 87, wherein the aromatase inhibitor is selected from at least one of non-steroidal and reversible aromatase inhibitors.

95. The use as claimed in claim 87, wherein the aromatase inhibitor is non-reversible.

96. The use as claimed in claim 87, wherein the medicament is for oral administration.

97. The use as claimed in claim 87, wherein the mood disorder is loss of libido.

98. The use as claimed in claim 87, wherein the mood disorder is depression.

99. A pharmaceutical composition comprising an aromatase inhibitor and an estrogen.

100. A pharmaceutical preparation for the treatment of a male in need of hormone replacement therapy, the preparation comprising a plurality of doses of an aromatase inhibitor and a plurality of doses of an estrogen.

101. A pharmaceutical preparation for the treatment of a male in need of treatment for a mood disorder, the preparation comprising a plurality of doses of an aromatase inhibitor and a plurality of doses of an estrogen.

102. A pharmaceutical preparation as claimed in claim 93, wherein the mood disorder is loss of libido.

103. A pharmaceutical preparation as claimed in claim 93, wherein the mood disorder is depression.
